(19) United States
(12) Patent Application Publication
(10) Pub. No.: US 2026/0029410 A1
(43) Pub. Date: Jan. 29, 2026

(54) PROGNOSTIC AND DIAGNOSTIC METHODS FOR RISK OF ACUTE KIDNEY INJURY

(71) Applicant: PREVENCIO, INC., Kirkland, WA (US)

(72) Inventors: Rhonda Fay RHYNE, Kirkland, WA (US); Craig Agamemnon MAGARET, Seattle, WA (US); Grady BARNES, Grayslake, IL (US); James Louis JANUZZI, JR., Wellesley, MA (US); John STROBECK, Allendale, NJ (US)

(21) Appl. No.: 19/058,816

(22) Filed: Feb. 20, 2025

Related U.S. Application Data
(63) Continuation of application No. 17/290,750, now abandoned.
(60) Provisional application No. 62/755,272, filed on Nov. 2, 2018.

(51) Int. Cl. G01N 33/68; G01N 33/62; G01N 33/70

(52) U.S. Cl. CPC G01N 33/6893; G01N 33/62; G01N 33/70; G01N 2333/4737; G01N 2333/70596; G01N 2333/745; G01N 2800/34

(57) ABSTRACT

Compositions and methods are provided for diagnosis and/or prognosis of acute kidney injury risk following medical procedures in a subject. In some embodiments, the method includes measuring and comparing the level of particular proteins to other proteins. In other embodiments, the method includes measuring proteins levels with clinical variable information and comparing this composite with the composite of other protein levels with clinical variable information.

### CROSS-REFERENCE TO RELATED APPLICATION

This application claims priority to U.S. Provisional Application Ser. No. 62/755,272, filed on Nov. 2, 2018. The Provisional application is incorporated by reference in its entirety.

### FIELD

The present disclosure relates protein marker panels, assays, and kits and methods for determining the diagnosis, monitoring, and/or prognosis of acute kidney injury following procedures or interventions in a patient.

### BACKGROUND

Acute kidney injury (AKI) following interventional procedures has substantial impact on patient management and prognosis.

The incidence of acute kidney injury (AKI) following angiographic procedures varies widely due to different definition criteria. Furthermore, the presence of co-morbidities including diabetes, chronic kidney disease (CKD), and heart failure (HF) further increase risk of AKI development [1]. Causes of peri-procedural AKI after angiographic procedures include contrast-induced AKI and, less commonly, atheroembolism. Regardless of cause, AKI has substantial impact on patient management and prognosis; it has been associated with worsening of CKD, requirement for dialysis, prolonged hospital stay, and higher mortality rates and health care costs [2]. Development of AKI is diagnosed using changes in serum creatinine or estimated glomerular filtration rate (eGFR). However, these measures of kidney function are only modestly useful for accurate prediction of risk for kidney injury [3]. This has led to interest in developing tools to accurately prospectively predict incident AKI and in some cases, earlier than when changes in creatinine or eGFR may occur [4-6]. In recent studies, machine learning was employed to develop models that predicted AKI in hospitalized patients with excellent accuracy [7-8]; and similarly, genomic and proteomic characterization of AKI has been undertaken with varying results. [9-11]

Standard measures of kidney function are only modestly useful for accurate prediction of risk for AKI. A need therefore exists for simple and reliable methods to improve the prognosis and/or monitoring of procedural acute kidney injury and associated outcomes.

### SUMMARY

In an aspect, provided herein are methods of determining acute kidney injury risk in a subject. The methods include providing a biological sample from a subject suspected of having acute kidney injury risk, applying the biological sample to an analytical device that is programmed to detect the concentration of at least two protein markers in the sample, normalize the concentrations against a quantification standard, and transform the normalized concentrations. The methods include optionally determining the status of at least one clinical variable or measurement, calculating a prognostic score using an algorithm based on the transformed, normalized concentration of protein markers and optionally, the status of the clinical variable or measurement, classifying the score as a positive, intermediate, or negative result, and determining acute kidney injury risk in the subject as indicated by the prognostic score. The protein markers are selected from Table 1. The optional clinical variable and/or measurement is selected from Table 2.

In an aspect, provided herein are methods of administering a therapeutic intervention to a subject suspected of having acute kidney injury risk. The methods include (i) determining the subject's protein marker profile for a panel of at least two protein markers selected from Table 1; (ii) optionally, determining the status of at least one clinical variable or measurement for the subject, where the clinical variable or measurement is selected from Table 2; (iii) assigning a score to the subject based on the protein marker profile in (i) and optionally the clinical value status in (ii); and (iv) administering to the subject a therapeutic intervention based on the positive, intermediate or negative score. Provided in the methods herein, the score is selected from positive, intermediate, and negative, and the score is algorithmically derived from the normalized and mathematically transformed concentrations of protein markers in the subject's sample and optionally, the status of at least one clinical variable or measurement.

In an aspect, provided herein are methods of monitoring acute kidney injury risk in a subject. The methods include providing a biological sample from a subject undergoing a contrast imaging procedure with risk of acute kidney injury or a subject suspected of having or had acute kidney injury risk, applying the biological sample to an analytical device that is programmed to detect the concentration of at least two protein markers in the sample, normalize the concentrations against a quantification standard, and transform the normalized concentrations. The methods include optionally determining the status of at least one clinical variable or measurement, calculating a prognostic score using an algorithm based on the transformed, normalized concentration of protein markers and optionally, the status of the clinical variable or measurement, classifying the score as a positive, intermediate, or negative result, and determining acute kidney injury risk in the subject as indicated by the prognostic score. The protein markers are selected from Table 1. The optional clinical variable and/or measurement is selected from Table 2.

In an aspect, provided herein are methods of detecting two or more protein markers in a subject having diabetes type 2 and/or is suspected of having acute kidney injury risk. The methods include selecting a subject that has diabetes type 2 and/or is suspected of having acute kidney injury risk, providing a biological sample from the subject, applying the biological sample to an analytical device, and detecting the concentration of at least two protein markers selected from Table 1.

In an aspect, provided herein are panels for the prognosis of acute kidney injury. The panel includes target-binding agents that bind at least two protein markers selected from Table 1. The panel optionally includes at least one clinical variable or measurement selected from Table 2.

In an aspect, provided herein are panels for the prognosis of acute kidney injury. The panels includes target-binding agents that bind protein markers for CD5 antigen-like, C reactive protein, Factor VII, kidney injury molecule 1, N-terminal prohormone of brain natriuretic peptide, and/or osteopontin and includes the clinical measurement of blood urea nitrogen creatinine ratio and, optionally, the clinical variable of history of diabetes mellitus type 2.

In an aspect, provided herein are panels for the prognosis of acute kidney injury. The panel includes target-binding agents that bind protein markers for CD5 antigen like, C reactive protein, Factor VII, and osteopontin and includes the clinical measurement of blood urea nitrogen creatinine ratio and the clinical variable of history of diabetes mellitus type 2.

In an aspect, provided herein are panels for the prognosis of acute kidney injury. The panel includes target-binding agents that bind protein markers for CD5 antigen like, C reactive protein, Factor VII, kidney injury molecule 1, and osteopontin and includes the clinical measurement of blood urea nitrogen:creatinine ratio and the clinical variable of history of diabetes mellitus type 2.

In an aspect, provided herein are panels for the prognosis of acute kidney injury. The panel includes target-binding agents that bind protein markers for C reactive protein, kidney injury molecule 1, and osteopontin and includes the clinical measurement of blood urea nitrogen:creatinine ratio and the clinical variable of history of diabetes mellitus type 2.

In an aspect, provided herein are panels for the prognosis of acute kidney injury. The panel includes target-binding agents that bind protein markers for C Reactive Protein and N-terminal prohormone of brain natriuretic peptide and includes the clinical measurement of blood urea nitrogen:creatinine ratio.

### BRIEF DESCRIPTION OF THE DRAWINGS

FIG. 1 shows a procedural acute kidney injury risk prediction model receiver operating characteristic curve. AUC=area under the receiver operating characteristic curve, Sn=sensitivity, Sp=specificity, PPV=positive predictive value, NPV=negative predictive value, ACC=accuracy. The receiver operating characteristic curve is for the Prevencio AKI panel AKI 026e (as described in Example 1) (N=889) to prognose the risk of AKI, and/or monitoring AKI progression. The panel had a robust cross-validated area under the curve (AUC) of 0.79, and an in-sample AUC of 0.816 rounded up to 0.82.

FIG. 2 shows a procedural acute kidney injury risk prediction model receiver operating characteristic curve. AUC=area under the receiver operating characteristic curve, Sn=sensitivity, Sp=specificity, PPV=positive predictive value, NPV=negative predictive value, ACC=accuracy. The receiver operating characteristic curve is for the Prevencio AKI panel AKI 027e (N=889) to prognose the risk of AKI, and/or monitoring AKI progression. The panel had a robust cross-validated area under the curve (AUC) of 0.78 and an in-sample AUC of 0.816 rounded up to 0.82.

FIG. 3 shows a procedural acute kidney injury risk prediction model receiver operating characteristic curve. AUC=area under the receiver operating characteristic curve, Sn=sensitivity, Sp=specificity, PPV=positive predictive value, NPV=negative predictive value, ACC=accuracy. The receiver operating characteristic curve is for the Prevencio AKI panel AKI 032e (N=889) to prognose the risk of AKI, and/or monitoring AKI progression. The panel had a robust cross-validated area under the curve (AUC) of 0.45 and an in-sample AUC of 0.765 rounded up to 0.77.

FIG. 4 shows a procedural acute kidney injury risk prediction model receiver operating characteristic curve. AUC=area under the receiver operating characteristic curve, Sn=sensitivity, Sp=specificity, PPV=positive predictive value, NPV=negative predictive value, ACC=accuracy. The receiver operating characteristic curve is for the Prevencio AKI panel AKI 052e (N=889) to prognose the risk of AKI, and/or monitoring AKI progression. The panel had a robust cross-validated area under the curve (AUC) of 0.75 and an in-sample AUC of 0.761 rounded up to 0.76.

### DETAILED DESCRIPTION

The practice of the technology described herein will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. [12-31].

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2, or 1% to a reference quantity, level, value, concentration, measurement, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The terms “disease” or “condition” refer to a state of being or health status of a patient or subject capable of being treated with the compounds or methods provided herein. The disease may be a cardiovascular disease. The disease may be an inflammatory disease. In some instances, the condition is acute kidney injury. In some instances, the disease is diabetes mellitus type 2.

As used herein, the term “diagnosis” refers to an identification or likelihood of the presence of acute kidney injury or outcome in a subject.

As also used herein, the term “prognosis” refers to the likelihood or risk of a subject developing a particular outcome or particular event such as a risk of acute kidney injury.

As used herein, a “biological sample” encompasses essentially any sample type that can be used in a diagnostic or prognostic method described herein. The biological sample may be any bodily fluid, tissue or any other sample from which clinically relevant protein or chemical compound marker concentrations may be determined. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polypeptides or proteins. The term “biological sample” encompasses a clinical sample, but also, in some instances, includes blood, serum, plasma, urine, cerebral spinal fluid, biological fluid, and tissue samples. The sample may be pretreated as necessary by dilution in an appropriate buffer solution or concentrated, if desired. Any of a number of standard aqueous buffer solutions, employing one of a variety of buffers, such as phosphate, Tris, or the like, preferably at physiological pH can be used.

“Treating” or “treatment” as used herein (and as well understood in the art) broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

The term “prevent” refers to a decrease in the occurrence of disease symptoms in a patient. The prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition. Non-limiting examples include humans, other mammals, bovines, rats, mice, dogs, monkeys, goat, sheep, cows, deer, and other non-mammalian animals. In some embodiments, a patient is human.

“Control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples). In some instances, the control is a quantification standard used as a reference for assay measurements. The quantification standard may be a synthetic protein marker, a recombinantly expressed purified protein marker, a purified protein marker isolated from its natural environment, a protein fragment, a synthesized polypeptide, or the like.

The term “cardiovascular disease” refers to a class of diseases that involve the heart or blood vessels. Cardiovascular disease includes, but is not limited to, coronary artery diseases (CAD), myocardial infarction (commonly known as a heart attack), stroke, hypertensive heart disease, rheumatic heart disease, cardiomyopathy, congestive heart failure, cardiac arrhythmias (i.e., atrial fibrillation, ventricular tachycardia, etc.), cerebrovascular disease, peripheral arterial disease, aortic valve stenosis, and arterial thrombosis.

The term “cardiovascular event” as used herein denotes a variety of adverse outcomes related to the cardiovascular system. These events include, but are not limited to peripheral limb amputation, peripheral revascularization, myocardial infarct, heart failure, stroke, and cardiovascular death.

The term “acute kidney injury” refers to an abrupt loss of kidney function. Generally, it occurs because of damage to the kidney tissue caused by decreased kidney blood flow (kidney ischemia) from any cause (e.g., low blood pressure), exposure to substances harmful to the kidney, such as dye used in diagnostic and/or procedural catheterizations, an inflammatory process in the kidney, or an obstruction of the urinary tract that impedes the flow of urine. The causes of acute kidney injury are commonly categorized into pre-renal, intrinsic, and post-renal. Acute kidney injury occurs in up to 30% of patients following cardiac surgery. Mortality increases by 60-80% in post-cardiopulmonary bypass patients who go on to require renal replacement therapy. AKI may lead to a number of complications, including metabolic acidosis, high potassium levels, uremia, changes in body fluid balance, and effects on other organ systems, including death. People who have experienced AKI may have an increased risk of chronic kidney disease in the future. Management includes treatment of the underlying cause and supportive care, such as renal replacement therapy.

As described herein, the terms “marker”, “protein marker”, “polypeptide marker”, and “biomarker” are used interchangeably throughout the disclosure. As used herein, a protein marker refers generally to a protein or polypeptide, the level or concentration of which is associated with a particular biological state, particularly a state associated with a cardiovascular disease, event or outcome. Panels, assays, kits and methods of the present disclosure may comprise antibodies, binding fragments thereof or other types of target-binding agents, which are specific for the protein marker described herein.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. In various embodiments, detecting the concentrations of naturally occurring protein marker proteins in a biological sample is contemplated for use within diagnostic, prognostic, or monitoring methods disclosed herein. The term also includes fusion proteins, including, but not limited to, naturally occurring fusion proteins with a heterologous amino acid sequence, fusions with heterologous and homologous leader sequences, with or without N-terminal methionine residues; immunologically tagged proteins; and the like. The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “antibody” herein is used in the broadest sense and specifically covers, but is not limited to, monoclonal antibodies, polyclonal antibodies, multi-specific antibodies (e.g., bispecific antibodies) formed from at least two intact antibodies, single chain antibodies (e.g., scFv), and antibody fragments or other derivatives, so long as they exhibit the desired biological specificity. The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “monoclonal antibody” as used herein refers to an antibody obtained from a population of substantially homogeneous antibodies, i.e., the individual antibodies comprising the population are identical except for possible naturally occurring mutations that can be present in minor amounts. In certain specific embodiments, the monoclonal antibody is an antibody specific for a protein marker described herein.

Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. [22], or may be made by recombinant DNA methods (see, e.g., U.S. Pat. No. 4,816,567), or any other suitable methodology known and available in the art. The “monoclonal antibodies” may also be isolated from phage antibody libraries using the techniques described in Clackson et al. [23] and Marks et al. [24], for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity and/or specificity [25-26]. Methods of making chimeric antibodies are known in the art.

An “isolated” antibody is one that has been identified and separated and/or recovered from a component of its natural environment. Contaminant components of its natural environment are materials that would interfere with diagnostic or prognostic uses for the antibody, and may include enzymes, hormones, and other proteinaceous or non-proteinaceous solutes. In specific embodiments, the antibody will be purified to greater than 95% by weight of antibody, e.g., as determined by the Lowry method, and most preferably more than 99% by weight.

The terms “detectably labeled antibody” refers to an antibody (or antibody fragment) which retains binding specificity for a protein marker described herein, and which has an attached detectable label. The detectable label can be attached by any suitable means, e.g., by chemical conjugation or genetic engineering techniques. Methods for production of detectably labeled proteins are well known in the art. Detectable labels may be selected from a variety of such labels known in the art, including, but not limited to, haptens, radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, and luciferase/luciferin)), methods for labeling antibodies, and methods for using labeled antibodies are well known in the art [27].

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, immunoassays are routinely used to select antibodies specifically immunoreactive with a protein.

An example immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “V_{H},” or “VH” refer to the variable region of an immunoglobulin heavy chain, including an Fv, scFv, dsFv or Fab, while the terms “variable light chain,” “V_{L}” or “VL” refer to the variable region of an immunoglobulin light chain, including of an Fv, scFv, dsFv or Fab.

“Functional fragments” of antibodies can also be used and include those fragments that retain sufficient binding affinity and specificity for a protein marker to permit a determination of the level of the protein marker in a biological sample. In some cases, a functional fragment will bind to a protein marker with substantially the same affinity and/or specificity as an intact full chain molecule from which it may have been derived. Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen. As appreciated by one of skill in the art, various antibody fragments can be obtained by a variety of methods, for example, digestion of an intact antibody with an enzyme, such as pepsin, or de novo synthesis. Antibody fragments are often synthesized de novo either chemically or by using recombinant DNA methodology. Thus, the term antibody, as used herein, includes antibody fragments produced by the modification of whole antibodies, or those synthesized de novo using recombinant DNA methodologies (e.g., single chain Fv) or those identified using phage display libraries.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the disclosure include humanized and/or chimeric monoclonal antibodies.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form.

A “substantially isolated” or “isolated” substance is one that is substantially free of its associated surrounding materials in nature. By substantially free is meant at least 50%, preferably at least 70%, more preferably at least 80%, and even more preferably at least 90% free of the materials with which it is associated in nature. As used herein, “isolated” can refer to polynucleotides, polypeptides, antibodies, cells, samples, and the like.

As used herein, “adiponectin” refers to a protein involved in regulating glucose as well as fatty acid breakdown. It is also referred to as GBP-28, apM1, AdipoQ, and Acrp30. Adiponectin is a 244-amino-acid peptide secreted by adipose tissue, whose roles include the regulation of glucose and fatty acid metabolism.

As used herein, “angiopoietin 1” refers to is a type of angiopoietin and is encoded by the gene ANGPT1. Angiopoietins are proteins with important roles in vascular development and angiogenesis. All angiopoietins bind with similar affinity to an endothelial cell-specific tyrosine-protein kinase receptor. The protein encoded by this gene is a secreted glycoprotein that activates the receptor by inducing its tyrosine phosphorylation. It plays a critical role in mediating reciprocal interactions between the endothelium and surrounding matrix and mesenchyme. The protein also contributes to blood vessel maturation and stability, and may be involved in early development of the heart

As used herein, “apolipoprotein(a)”, also referred to as “apo(a)”, is the main constituent of lipoprotein(a) (Lp(a)). Apolipoprotein(a) has serine proteinase activity and is capable of auto proteolysis. Apolipoprotein(a) inhibits tissue-type plasminogen activator 1. Apolipoprotein(a) is known to be proteolytically cleaved, leading to the formation of the so-called mini-Lp(a). Apolipoprotein(a) fragments accumulate in atherosclerotic lesions, where they may promote thrombogenesis.

As used herein, “apolipoprotein A-II” refers to an apolipoprotein found in high-density lipoprotein (HDL) cholesterol in plasma. Apolipoprotein (apo) A-II is the second major apo of high-density lipoproteins. Results suggest that enrichment of apo A-II in high-density lipoprotein particles may have athero-protective effects, although its exact mechanism is unclear. Apo A-II may become a target for the treatment of atherosclerosis.

As used herein, “apolipoprotein C-I” is a protein component of lipoproteins normally found in the plasma and responsible for the activation of esterified lecithin cholesterol and in removal of cholesterol from tissues.

As used herein, “angiotensin converting enzyme” or “ACE” refers to a central component of the renin-angiotensin system (RAS), which controls blood pressure by regulating the volume of fluids in the body. It converts the hormone angiotensin I to the active vasoconstrictor angiotensin II.

As used herein, “blood urea nitrogen” or “BUN” refers to a medical test that measures the amount of urea nitrogen found in blood. The liver produces urea in the urea cycle as a waste product of the digestion of protein.

As used herein, “creatinine” refers to a by-product of everyday muscle contraction while blood urea nitrogen measures the amount of urea nitrogen, a by-product of the urea cycle that breaks down amino acids, in the blood.

As used herein, “blood urea nitrogen to creatinine ratio” or “BCR” is a common laboratory test to help diagnose AKI (Mayo Clinic (2016) Blood urea nitrogen (BUN) test. https://www.mayoclinic.org/tests-procedures/blood-urea-nitrogen/about/pac-20384821).

As used herein, “CD5 antigen-like” or “CD5L”, also known as “apoptosis inhibitor of macrophage”, is a protein that is expressed in inflamed tissues. In HART AKI, decreased concentrations of CD5L correlated with an increased risk for AKI.

As used herein, “C reactive protein” or “CRP” is an acute-phase reactant protein that responds rapidly to inflammation.

As used herein, “carcinoembryonic antigen related cell adhesion molecule 1” or “biliary glycoprotein” or “CEACAM1” also known as “CD66a” (Cluster of Differentiation 66a), is a human glycoprotein that mediates cell adhesion via homophilic as well as heterophilic binding to other proteins of the subgroup.

As used herein, “cystatin”, also known as “Cystatin C” or “cystatin 3” (formerly “gamma trace”, “post-gamma-globulin”, or “neuroendocrine basic polypeptide”), is a protein encoded by the CST3 gene, is mainly used as a biomarker of kidney function. Recently, it has been studied for its role in predicting new-onset or deteriorating cardiovascular disease. Cystatin belongs to the type 2 cystatin gene family.

As used herein, “decorin”, also known as “PG40” and “PGS2”, is a protein, which belongs to the small leucine-rich proteoglycan family. It regulates assembly of the extracellular collagen matrix.

As used herein, “eotaxin 1”, also known as “C-C motif chemokine 11” and “eosinophil chemotactic protein” is a small cytokine belonging to the CC chemokine family.

As used herein, “ENRAGE”, also known as “extracellular newly identified receptor for advanced glycation end-products binding protein”, has been implicated in various inflammatory diseases and/or states including cardiovascular disease

As used herein, “Factor VII”, also known as “blood-coagulation factor VIIa”, “activated blood coagulation factor VII”, or “proconvertin” is one of the proteins that causes blood to clot in the coagulation cascade. Factor VII is a serine protease that, once activated, catalyzes the activation of factor X in the coagulation pathway [28].

As used herein, “ferritin” is a universal intracellular protein that stores iron and releases it in a controlled fashion.

As used herein, fetuin A, also known as “alpha-2-HS-glycoprotein” or “AHSG” is a protein that belongs to the fetuin class of plasma binding proteins and is more abundant in fetal than adult blood.

As used herein, “follicle stimulating hormone” or “FSH”, is a gonadotropin, a glycoprotein polypeptide hormone. FSH is synthesized and secreted by the gonadotropic cells of the anterior pituitary gland and regulates the development, growth, pubertal maturation, and reproductive processes of the body.

As used herein, “growth hormone”, also known as “somatotropin” or as “human growth hormone” or “hGH” in its human form, is a peptide hormone that stimulates growth, cell reproduction, and cell regeneration in humans and other animals. It is thus important in human development. It is a type of mitogen specific only to certain kinds of cells. GH is a stress hormone that raises the concentration of glucose and free fatty acids.

As used herein, “immunoglobulin M” or “IgM” is one of several forms of antibody that are produced by vertebrates. IgM is the largest antibody, and it is the first antibody to appear in the response to initial exposure to an antigen.

As used herein, “intercellular adhesion molecule 1” also known as “ICAM-1” and “CD54” or “Cluster of Differentiation 54” is a cell surface glycoprotein, which is typically expressed on endothelial cells and cells of the immune system. It binds to integrins of type CD11a/CD18, or CD11b/CD18.

As used herein, “interferon gamma induced protein 10”, also known as “CXCL10”, “IP-10” and “10 kDa interferon-gamma-induced protein”, is considered a member of the CXC chemokine and is induced in a variety of cells in response to IFN-gamma. It has proven to be a valid protein marker for the development of cardiovascular disease, including heart failure and left ventricular dysfunction, suggesting an underlining pathophysiological relation with the development of adverse cardiac remodeling.

As used herein, “interleukin-1 receptor antagonist” or “IL-RA” also known as “interleukin 1 inhibitor” or “IL-1 inhibitor”, refers to a protein that is a member of the interleukin 1 cytokine family. IL-RA is secreted by various types of cells including immune cells, epithelial cells, and adipocytes, and is a natural inhibitor of the pro-inflammatory effect of IL1β. This protein inhibits the activities of interleukin 1, alpha (IL1A) and interleukin 1, beta (IL1B), and modulates a variety of interleukin 1 related immune and inflammatory responses.

As used herein, “interleukin-8”, also known as “IL8”, “neutrophil chemotactic factor”, “chemokine ligand 8”, and “CXCL8”, is a chemokine produced by macrophages and other cell types such as epithelial cells, airway smooth muscle cells, and endothelial cells. It induces chemotaxis in target cells, primarily neutrophils but also other granulocytes, causing them to migrate toward the site of infection. IL-8 also induces phagocytosis once they have arrived. IL-8 is also known to be a potent promoter of angiogenesis. In target cells, IL-8 induces a series of physiological responses required for migration and phagocytosis, such as increases in intracellular Ca²⁺, exocytosis (e.g. histamine release), and the respiratory burst.

As used herein, “interleukin-18” also known as “IL-18”, is a proinflammatory cytokine produced by macrophages and other cells. IL-18 works by binding to the interleukin-18 receptor, and together with IL-12, it induces cell-mediated immunity following infection with microbial products like lipopolysaccharide (LPS). After stimulation with IL-18, natural killer (NK) cells and certain T cells release another important cytokine called interferon-γ (IFN-γ) or type II interferon that plays an important role in activating the macrophages or other cells.

As used herein, “interleukin-23”, also known as “IL-23”, is a heterodimeric cytokine composed of an IL12B (IL-12p40) subunit (that is shared with IL12) and the IL23A (IL-23p19) subunit. It has been shown to facilitate development of inflammation in numerous other models of immune pathology where IL-12 had previously been implicated including models of arthritis, intestinal inflammation and psoriasis.

As used herein, “kidney injury molecule 1”, also known as “kidney injury molecule-1” and “KIM-1” is a type I cell membrane glycoprotein that serves as a receptor for oxidized lipoproteins and plays a functional role in the kidney. KIM-1 is a proximal renal tubular marker, concentrations of which have been linked to acute kidney injury.

As used herein, “lipoprotein(a)”, also known as “Lp(a)”, is a subclass of lipoproteins. It a consists of an LDL-like particle and the specific apolipoprotein(a) (apo(a)), which is covalently bound to the apolipoprotein B of the LDL like particle. Lp(a) as a risk factor for atherosclerotic cardiovascular diseases.

As used herein, “matrix metalloproteinase 7”, also known as “MMP-7”, “Matrilysin”, “pump-1 protease (PUMP-1)”, or “uterine metalloproteinase”, is an enzyme in humans with a primary role to break down extracellular matrix.

As used herein, “matrix metalloproteinase 9”, also known as “MMP-9”, “92 kDa type IV collagenase”, “92 kDa gelatinase”, and “gelatinase B” or “GELB”, is a matrixin, a class of enzymes that belong to the zinc-metalloproteinase family involved in the degradation of the extracellular matrix. Proteins of the matrix metalloproteinase (MMP) family are involved in the breakdown of extracellular matrix in normal physiological processes, such as embryonic development, reproduction, angiogenesis, bone development, wound healing, cell migration, learning and memory, as well as in pathological processes, such as arthritis, intracerebral hemorrhage, and metastasis.

As used herein, “matrix metalloproteinase 9 Total”, also known as “MMP-9 Total”, refers to a combination and/or ratio of matrix metalloproteinase 9 (MMP9) and tissue inhibitor of metalloproteinase 1 (TIMP-1). Matrix metalloproteinase 9, also known as MMP-9, 92 kDa type IV collagenase, 92-kDa gelatinase, and gelatinase B or GELB, is a matrixin, a class of enzymes that belong to the zinc-metalloproteinase family involved in the degradation of the extracellular matrix. Proteins of the matrix metalloproteinase (MMP) family are involved in the breakdown of extracellular matrix in normal physiological processes, such as embryonic development, reproduction, angiogenesis, bone development, wound healing, cell migration, learning and memory, as well as in pathological processes, such as arthritis, intracerebral hemorrhage, and metastasis. TIMP-1 is a glycoprotein that is expressed from several tissues. It is a member of the TIMP family and is a natural inhibitor of the matrix metalloproteinases (MMPs), a group of peptidases involved in degradation of the extracellular matrix. In addition to its inhibitory role against most of the known MMPs, the encoded protein is able to promote cell proliferation in a wide range of cell types, and may have an anti-apoptotic function. TIMP-1 has been associated plaque rupture and adverse cardiovascular events.

As used herein, “midkine”, also known as “neurite growth-promoting factor 2” or “NEGF2”, refers to a basic heparin-binding growth factor of low molecular weight and forms a family with pleiotrophin. Midkine is a heparin-binding cytokine/growth factor with a molecular weight of 13 kDa.

As used herein, “monokine induced by gamma interferon”, also known as “MIG” or “CXCL9”, is a small cytokine belonging to the family of CXC chemokines. It is a T-cell chemoattractant and has been associated with worsening left ventricular dysfunction and symptomatic cardiovascular disease.

As used herein, “myeloid progenitor inhibitory factor 1” also known as Chemokine (C-C motif) ligand 23, “CCL23”, “Macrophage inflammatory protein 3”, and “MIP-3” is a small cytokine belonging to the CC chemokine family. It is predominantly expressed in lung and liver tissue, but is also found in bone marrow and placenta. It is also expressed in some cell lines of myeloid origin.

As used herein, “myeloperoxidase” also known as “MPO” is a white blood cell-derived inflammatory enzyme that measures disease activity from the luminal aspect of the arterial wall. When the artery wall is damaged, or inflamed, myeloperoxidase is released by invading macrophages where it accumulates. Myeloperoxidase mediates the vascular inflammation that propagates plaque formation and activates protease cascades that are linked to plaque vulnerability.

As used herein, “myoglobin” is an iron- and oxygen-binding protein found in the muscle tissue of vertebrates in general and in almost all mammals. Myoglobin is released from damaged muscle tissue (rhabdomyolysis), which has very high concentrations of myoglobin. The released myoglobin is filtered by the kidneys but is toxic to the renal tubular epithelium and so may cause acute kidney injury. It is not the myoglobin itself that is toxic (it is a protoxin) but the ferrihemate portion that is dissociated from myoglobin in acidic environments (e.g., acidic urine, lysosomes). Myoglobin is a sensitive marker for muscle injury, making it a potential marker for heart attack in patients with chest pain.

As used herein, “N-terminal prohormone of brain natriuretic peptide” or “NT-PBNP” is also known as “NT-proBNP” or “BNPT” and refers to an N-terminal inactive protein that is cleaved from proBNP to release brain natriuretic peptide.

As used herein, “osteopontin” or “OPN”, also known as “bone sialoprotein I”, “BSP-1”, “BNSP”, “early T-lymphocyte activation”, “ETA-1”, “secreted phosphoprotein 1”, “SPP1”, “2ar”, “Rickettsia resistance”, or “Ric”, refers to a glycoprotein (small integrin binding ligand N-linked glycoprotein) first identified in osteoblasts. It includes all isoforms and post-translational modifications. It is synthesized and secreted in many tissues including bone, cardiac tissues, and kidneys. In normal adult human kidneys, OPN is highly expressed in the loop of Henle [29]. OPN can be upregulated during inflammation and is involved in the recruitment of macrophages to the site of inflammation [30].

As used herein, “pulmonary surfactant associated protein D”, also referred to as surfactant, pulmonary-associated protein D, or SP-D or SFTPD, is a protein that contributes to the lung's defense against inhaled microorganisms, organic antigens and toxins.

As used herein, “resistin” also known as “adipose tissue-specific secretory” factor or “ADSF” or “C/EBP-epsilon-regulated myeloid-specific secreted cysteine-rich protein” or “XCP1” is a cysteine-rich adipose-derived peptide hormone. Resistin increases the production of LDL in human liver cells and degrades LDL receptors in the liver. As a result, the liver is less able to clear ‘bad’ cholesterol from the body. Resistin accelerates the accumulation of LDL in arteries, increasing the risk of heart disease. Resistin adversely impacts the effects of statins, the main cholesterol-reducing drug used in the treatment and prevention of cardiovascular disease.

As used herein, “serotransferrin”, also known as “transferrin”, is an abundant blood plasma glycoprotein with a main function of binding and transporting iron throughout the body. In patients with cardiovascular disease, low concentrations of serotransferrin causes iron deficiency, which correlates with decreased exercise capacity and poor quality of life, and predicts worse outcomes.

As used herein, “stem cell factor”, also known as “SCF”, “KIT-ligand”, “KL”, and “steel factor”, is a cytokine that binds to the c-KIT receptor (CD117). SCF can exist as both a transmembrane protein and a soluble protein. This cytokine plays an important role in hematopoiesis (formation of blood cells), spermatogenesis, and melanogenesis.

As used herein, “Tamm Horsfall Urinary Glycoprotein” or “THP”, also known as “uromodulin”, is a glycoprotein that is the most abundant protein excreted in ordinary urine.

As used herein, “tissue inhibitor of metalloproteinase 1, also known as “TIMP-1” or “TIMP metallopeptidase inhibitor 1”, is a glycoprotein expressed in several tissues. It is a member of the TIMP family and is a natural inhibitor of the matrix metalloproteinases (MMPs), a group of peptidases involved in degradation of the extracellular matrix. In addition to its inhibitory role against most of the known MMPs, the encoded protein is able to promote cell proliferation in a wide range of cell types, and may have an anti-apoptotic function. TIMP-1 has been associated plaque rupture and adverse cardiovascular events.

As used herein, “T Cell Specific Protein RANTES”, also known as “RANTES”, “regulated on activation, normal T cell expressed and secreted”, “Chemokine (C-C motif) ligand 5”, or “CCL5”, is a protein that is chemotactic for T cells, eosinophils, and basophils, and plays an active role in recruiting leukocytes into inflammatory sites. With the help of particular cytokines (i.e., IL-2 and IFN-γ) that are released by T cells, CCL5 also induces the proliferation and activation of certain natural-killer (NK) cells to form CHAK (CC-Chemokine-activated killer) cells.

As used herein, “thyroxine binding globulin”, or “TBG” a globulin that binds thyroid hormones in circulation. It is one of three transport proteins (along with transthyretin and serum albumin) responsible for carrying the thyroid hormones thyroxine (T₄) and triiodothyronine (T₃) in the bloodstream.

As used herein, “transthyretin” or “TTR” is a transport protein in the serum and cerebrospinal fluid that carries the thyroid hormone thyroxine (T₄) and retinol-binding protein bound to retinol.

As used herein, “troponin”, also known as the “troponin complex”, is a complex of three regulatory proteins (troponin C, troponin I, and troponin T) that is integral to muscle contraction in skeletal muscle and cardiac muscle, but not smooth muscle. As used herein a troponin protein marker may identify each of these proteins individually, or in combination, and may be any level of sensitivity. An increased level of the cardiac protein isoform of troponin circulating in the blood has been shown to be a protein marker of heart disorders and heart stress, the most important of which is myocardial infarction. Raised troponin concentrations indicate cardiac muscle cell death as the molecule is released into the blood upon injury to the heart.

As used herein, “vascular cell adhesion molecule”, also known as “VCAM-1”, “VCAM”, “cluster of differentiation 106”, and “CD106”, is a cell adhesion molecule. The VCAM-1 protein mediates the adhesion of lymphocytes, monocytes, eosinophils, and basophils to vascular endothelium. It also functions in leukocyte-endothelial cell signal transduction, and it may play a role in the development of atherosclerosis and rheumatoid arthritis.

As used herein, “vitamin D binding protein”, also known as “gc-globulin” or “group-specific component”, belongs to the albumin gene family, together with human serum albumin and alpha-fetoprotein. It is a multifunctional protein found in plasma, ascetic fluid, and cerebrospinal fluid and on the surface of many cell types. It is able to bind the various forms of vitamin D including ergocalciferol (vitamin D₂) and cholecalciferol (vitamin D₃), the 25-hydroxylated forms (calcifediol), and the active hormonal product, 1,25-dihydroxyvitamin D (calcitriol). The major proportion of vitamin D in blood is bound to this protein. It transports vitamin D metabolites between skin, liver and kidney, and then on to the various target tissues.

As used herein, “von Willebrand Factor” or “vWF” is a blood glycoprotein involved in hemostasis. Its primary function is binding to other proteins, in particular factor VIII, cells, and molecules. It is important in platelet adhesion to wound sites, thus playing a major role in blood coagulation. It is not an enzyme and, thus, has no catalytic activity.

It will be understood by one skilled in the art that these and other protein markers disclosed herein (e.g., those set forth in Table 1) can be readily identified, made and used in the context of the present disclosure in light of the information provided herein.

As used herein, the term “score” refers to a binary, multilevel, or continuous result as it relates diagnostic or prognostic determinations. A score can be a positive, intermediate, or negative diagnostic score. A score can be a positive, intermediate, or negative prognostic score. One or multiple cutoffs can be used with the score to determine specific levels of risk. In embodiments, a score is algorithmically derived based on normalized and/or mathematically transformed values, such as protein concentrations, the presence/absence of clinical factors, vital statistics, or ratios of different factors. The algorithm, which generates the score, can be ratio-based, cut-off-based, linear or non-linear, including decision tree or rule-based models.

As used herein, the term “panel” refers to specific combination of protein markers and clinical markers used to determine a diagnosis, monitoring, and/or prognosis of a risk of acute kidney injury or outcome in a subject. The term “panel” may also refer to an assay comprising a set of protein markers used to determine a diagnosis, monitoring, and/or prognosis of a risk of acute kidney injury or outcome in a subject.

As further described herein, the “training set” is the set of patients or patient samples that are used in the process of training (i.e., developing, evaluating and building) the final diagnostic or prognostic model. The “validation set” is a set of patients or patient samples that are withheld from the training process, and are only used to validate the performance of the final diagnostic or prognostic model. If the set of patients or patient samples are limited in number, all available data may be used as a training set, or as an “in-sample” validation set.

As used herein, the term “normalized” refers to a type of transformation where the values are designed to fit a specific distribution, typically so that they are similar to the distributions of other variables. For example, for hypothetical proteins A and B, the raw concentration of protein A ranges from 0 to 500 and the raw concentration of Protein B ranges from 0 to 20,000, it is not trivial looking at the raw values to determine which one is “higher”. For instance, is 400 of Protein A higher than 15,000 of Protein B? By conducting a normalization process, the concentrations are rescaled so that they are on the same scale: centered at zero, with a variance of 1. Thus, it becomes a routine exercise to determine which one is higher because the normalized concentrations are comparable. Many learning algorithms work better on data that are normalized; otherwise, in this example for instance, Protein B might get more weight in the algorithm because it has higher values even if it were not empirically “higher”.

As used herein, the term “transformed” refers to a mathematical process applied to a result, regardless of the input or output value. For example, it may include taking protein concentrations and calculating the base-10 logarithm from original values, reflecting a “log-transformation”.

### Protein Markers

Certain illustrative protein markers provided herein can be found listed in Table 1. Based on the information therein, the skilled artisan can readily identify, select and implement a protein marker or protein marker combination in accordance with the methods provided herein.

In embodiments, at least 2, at least 3, at least 4, or at least 5 protein markers from Table 1 are used in the methods and panels provided herein. In an embodiment, two proteins from Table 1 are selected. In an embodiment, three proteins from Table 1 are selected. In an embodiment, four proteins from Table 1 are selected. In an embodiment, five proteins from Table 1 are selected. In an embodiment, six proteins from Table 1 are selected. In other embodiments, the number of protein markers employed can vary, and may include at least 6, 7, 8, 9, 10, or more. In still other embodiments, the number of protein markers can include at least 15, 20, 25, or more. Also, in some embodiments, one or more of the protein markers from Table 1 can be specifically excluded. For example, 1, 2, 3, 4, 5, 6, 7 or more of the specific protein markers can be excluded from some embodiments, in any combination.

In certain specific embodiments, the protein markers used herein include those listed in Table 1, particularly those that are associated with a p-value of less than 0.1, less than 0.05, less than 0.01 or less than 0.001.

In embodiments, the protein markers used in accordance with the present disclosure are selected from CD5 antigen like, C reactive protein, Factor VII, kidney injury molecule 1, N-terminal prohormone of brain natriuretic peptide, and osteopontin and used in conjunction with the clinical lab measure of blood urea nitrogen:creatinine ratio and/or the clinical variable of history of diabetes mellitus type 2.

In embodiments, the protein markers used in accordance with the present disclosure are selected from CD5 antigen like, C reactive protein, Factor VII, and osteopontin and used in conjunction with the clinical lab measure of blood urea nitrogen:creatinine ratio and the clinical variable of history of diabetes mellitus type 2. In some embodiments, one or more (any combination) of the above-listed protein markers can be specifically excluded from any of the embodiments and aspects described herein.

In still other embodiments, the protein markers used in accordance with the present disclosure are selected from CD5 antigen like, C-reactive protein, Factor VII, kidney injury molecule 1, and osteopontin and used in conjunction with the clinical lab measure of blood urea nitrogen:creatinine ratio and the clinical variable of history of diabetes mellitus type 2. In some embodiments, one or more (any combination) of the above-listed protein markers can be specifically excluded from any of the embodiments and aspects described herein.

In still other embodiments, the protein markers used in accordance with the present disclosure are selected from C-reactive protein, kidney injury molecule 1, and osteopontin and used in conjunction with the clinical lab measure of blood urea nitrogen:creatinine ratio and the clinical variable of history of diabetes mellitus type 2. In some embodiments, one or more (any combination) of the above-listed protein markers can be specifically excluded from any of the embodiments and aspects described herein.

In still other embodiments, the protein markers used in accordance with the present disclosure are selected from C-reactive protein and N-terminal prohormone of brain natriuretic peptide and used in conjunction with the clinical lab measure of blood urea nitrogen:creatinine ratio. In some embodiments, one or more (any combination) of the above-listed protein markers can be specifically excluded from any of the embodiments and aspects described herein.

In embodiments, as noted elsewhere herein, a protein as recited in Table 1 may be specifically excluded from the methods or panels described herein.

TABLE 1
is a list of proteins whose concentrations are diagnostic or prognostic of
procedural acute kidney injury in a patient.

| | |
|---|---|
| Adiponectin | Interleukin-18 binding protein |
| Alpha 1 Antitrypsin | Interleukin-23 |
| Alpha 2 Macroglobulin | Kidney Injury Molecule 1 |
| Angiopoietin 1 | Lectin Like Oxidized LDL |
| | Receptor 1 |
| Angiotensin Converting | Leptin |
| Enzyme | |
| Apolipoprotein(a) | Lipoprotein(a) (Lp(a)) |
| Apolipoprotein AI | Luteinizing Hormone |
| Apolipoprotein AII | Macrophage Colony Stimulating |
| | Factor 1 |
| Apolipoprotein B | Macrophage Inflammatory Protein |
| | 1 alpha |
| Apolipoprotein CI | Macrophage Inflammatory Protein |
| | 1 beta |
| Apolipoprotein CIII | Macrophage Inflammatory Protein |
| | 3 alpha |
| Apolipoprotein H | Matrix Metalloproteinase 1 |
| Beta 2 Microglobulin | Matrix Metalloproteinase 2 |
| Brain Derived Neurotrophic | Matrix Metalloproteinase 3 |
| Factor | |
| C Reactive Protein | Matrix Metalloproteinase 7 |
| Calbindin | Matrix Metalloproteinase 9 |
| Carbonic anhydrase 9 | Matrix Metalloproteinase 9 Total |
| Carcinoembryonic antigen | Matrix Metalloproteinase 10 |
| related cell adhesion | |
| molecule 1 | |
| CD5 Antigen like | Midkine |
| Cystatin | Monocyte Chemotactic Protein 1 |
| Decorin | Monocyte Chemotactic Protein 2 |
| E Selectin | Monocyte Chemotactic Protein 4 |
| ENRAGE | Monokine Induced by Gamma |
| | Interferon |
| Eotaxin 1 | Myeloid Progenitor Inhibitory |
| | Factor 1 |
| Factor VII | Myeloperoxidase |
| Fatty Acid Binding Protein | Myoglobin |
| Ferritin | N terminal prohormone of |
| | brain natriuretic peptide |
| Fetuin A | Osteopontin |
| Fibrinogen | Pancreatic Polypeptide |
| Follicle Stimulating Hormone | Plasminogen Activator Inhibitor 1 |
| Glucagon-like Peptide-1 | Platelet endothelial cell adhesion |
| | molecule |
| Granulocyte Macrophage | Prolactin |
| Colony Stimulating Factor | |
| Growth Hormone | Pulmonary and Activation |
| | Regulated Chemokine |
| Haptoglobin | Pulmonary surfactant-associated |
| | protein D |
| Immunoglobulin A | Resistin |
| Immunoglobulin M | Serotransferrin |
| Insulin | Serum Amyloid P Component |
| Intercellular Adhesion | Stem Cell Factor |
| Molecule-1 | |
| Interferon gamma | T-Cell-Specific Protein RANTES |
| Interferon-gamma-Induced- | Tamm Horsfall Urinary |
| Protein 10 | Glycoprotein |
| Interleukin-1 alpha | Thrombomodulin |
| Interleukin-1 beta | Thrombospondin 1 |
| Interleukin-1 receptor | Thyroid Stimulating Hormone |
| antagonist | |
| Interleukin-2 | Thyroxine Binding Globulin |
| Interleukin-3 | Tissue Inhibitor of |
| | Metalloproteinases 1 (TIMP-1) |
| Interleukin-4 | Transthyretin |
| Interleukin-5 | Troponin |
| Interleukin-6 | Tumor Necrosis Factor alpha |
| Interleukin-6 receptor | Tumor Necrosis Factor beta |
| Interleukin-7 | Tumor necrosis factor receptor 2 |
| Interleukin-8 | Vascular Cell Adhesion Molecule 1 |
| Interleukin-10 | Vascular Endothelial Growth Factor |
| Interleukin-12 Subunit p40 | Vitamin D Binding Protein |
| Interleukin-12 Subunit p70 | Vitamin K-Dependent Protein S |
| Interleukin-15 | Vitronectin |
| Inter1eukin-17 | von Willebrand Factor |
| Interleukin-18 | |

In embodiments, the combination of proteins whose concentrations are correlated to the prognosis of a risk of procedural acute kidney injury risk and the nature of whether those protein concentrations are increased, decreased, or the same as compared to a healthy individual provides a subject's protein profile.

### Clinical Variables

As further described herein, the protein markers described herein can optionally be used in combination with certain clinical variables or measurement in order to provide for an improved diagnosis, monitoring, and/or prognosis of a risk of procedural acute kidney injury in a subject. As used herein, “optionally” refers to inclusion based on combinations of protein markers and their predictive value of a risk of procedural acute kidney injury or outcome when combined with a clinical variable factor. As used herein, “clinical variable” is used interchangeably with “clinical measure”, and “clinical measurement” and “lab measurement.” For example, illustrative clinical variables and measurements useful in the context of the present disclosure can be found listed in Table 2.

In embodiments, at least 1, at least 2, at least 3, at least 4, or at least 5 clinical variables from Table 2 are used in the methods and panels provided herein. In an embodiment, one clinical variable from Table 2 is selected. In an embodiment, two clinical variables from Table 2 are selected. In an embodiment, three clinical variables from Table 2 are selected. In an embodiment, four clinical variables from Table 2 are selected. In an embodiment, five clinical variables from Table 2 are selected. In other embodiments, the number of clinical variables employed can vary, and may include at least 6, 7, 8, 9, 10, or more.

In embodiments, the clinical variable(s) used in accordance with the present disclosure is history of diabetes type 2. In embodiments, the clinical measurement used in accordance with the present disclosure is blood urea nitrogen:creatinine ratio. In embodiments, the clinical variables used in accordance with the present disclosure are history of diabetes type 2 and blood urea nitrogen:creatinine ratio. In some embodiments, one or more (any combination) of the above-listed clinical variables can be specifically excluded from any of the embodiments and aspects described herein.

In embodiments, the presence/absence of clinical variables represented in binary form (e.g., history of diabetes mellitus type 2 (DM2), sex), and/or clinical variables in quantitative form (e.g., BMI, age, BUN:creatinine ratio) provide values that are entered into the diagnostic or prognostic model provided by the software, and the result is evaluated against one or more cutoffs to determine the diagnosis or prognosis.

In embodiments, one or more (any combination) of the clinical characteristic as recited in Table 2 may be specifically excluded from the methods and other embodiments described herein.

TABLE 2
is a list of clinical variables correlated to the diagnosis,
monitoring, and/or prognosis of procedural acute kidney injury.
Clinical Characteristics
Demographics
Age
Sex
Race
Vital Signs
Body Mass Index
Heart rate (beat/min)
Systolic BP (mmHg)
Diastolic BP (mmHg)
Medical History
Current smoker
Former smoker
History of atrial fibrillation/flutter
History of dyslipidemia
History of hypertension
History of coronary artery disease (CAD)
History of myocardial infarction (MI)
History of heart failure (HF)
History of peripheral artery disease (PAD)
History of COPD
History of diabetes mellitus, Type 1
History of diabetes mellitus, Type 2
History of any Diabetes
History of CVA/TIA
History of chronic kidney disease (CKD)
History of hemodialysis
History of angioplasty, (peripheral and/or coronary)
History of stent (peripheral and/or coronary)
History of CABG
History of coronary revascularization intervention
(coronary angioplasty, stent or bypass)
History of percutaneous coronary intervention
History of peripheral revascularization
History of percutaneous peripheral intervention
History of percutaneous peripheral angioplasty
(with or without stent)
History of resuscitation from sudden cardiac death
Family history of CAD
History of significant ventricular arrhythmia or suspected SCD
(not in the setting of acute MI)
Medications
ACE-I/ARB
Beta blocker
Aldosterone antagonist
Loop diuretics
Nitrates
CCB
Statin
Aspirin
Warfarin
Clopidogrel
Echocardiographic results
LVEF (%)
RSVP (mmHg)
Aortic valve area (AVA) (cm²)
Left ventricular internal diameter in end diastole (cm)
Posterior wall thickness of left ventricle (mm)
Inter-ventricular septal wall thickness (mm)
Left ventricular mass (grams)
Relative wall thickness (ratio of twice left ventricular diastolic wall
thickness to left ventricular end-diastolic dimension)
Mitral regurgitation (none, trace, mild, moderate, severe)
Aortic regurgitation (none, trace, mild, moderate, severe)
Tricuspid regurgitation (none, trace, mild, moderate, severe)
Peak velocity across aortic valve (cm/sec)
Left ventricular outflow tract velocity (cm/sec)
Peak gradient across aortic valve (mmHg)
Mean gradient across aortic valve (mmHg)
Stress test results
Ischemia on Scan
Ischemia on ECG
Angiography results
≥70% coronary stenosis
≥50% stenosis in at least one peripheral vessel
Lab Measures
Sodium
Blood urea nitrogen (mg/dL)
Creatinine (mg/dL)
Blood urea nitrogen:Creatinine Ratio
eGFR (median, CKDEPI)
Total cholesterol (mg/dL)
LDL cholesterol (mg/dL)
Glycohemoglobin (%)
Glucose (mg/dL)
HGB (mg/dL)
BP = blood pressure,
CAD = coronary artery disease,
MI = myocardial infarction,
HF = heart failure,
COPD = chronic obstructive pulmonary disease,
CVA/TIA = cerebrovascular accident/transient ischemic attack,
CKD = chronic kidney disease,
SCD = sudden cardiac death,
CABG = coronary artery bypass graft,
ACE-I/ARB = angiotensin converting enzyme inhibitor/angiotensin receptor blocker,
CCB = calcium channel blocker,
LVEF = left ventricular ejection fraction,
RVSP = right ventricular systolic pressure,
ECG = echocardiogram,
CKDEPI = Chronic Kidney Disease Epidemiology group (a standard for calculating eGFR),
eGFR = estimated glomerular filtration rate,
LDL = low density lipoprotein,
HGB = hemoglobin.

### Acute Kidney Injury

In an aspect, provided herein are methods of determining risk of procedural acute kidney injury in a subject. The methods include providing a biological sample from a subject suspected of having acute kidney injury risk, applying the biological sample to an analytical device that is programmed to detect the concentration of at least two protein markers in the sample calculate the concentrations against a quantification standard, and transform the normalized concentrations, and calculate a score using an algorithm The methods include optionally determining the status of at least one clinical variable or measurement, calculating a prognostic score using an algorithm based on the normalized, transformed concentrations of protein markers and optionally, the status of the clinical variable(s), classifying the prognostic score as a positive, intermediate, or negative result, and determining the risk of acute kidney injury in the subject as indicated by the prognostic score. The at least two protein markers are selected from Table 1. The optional clinical variable(s) or measurement(s) are selected from Table 2.

In an aspect, provided herein are methods of monitoring risk of procedural acute kidney injury in a subject. The methods include providing a biological sample from a subject undergoing a contrast imaging procedure with risk of acute kidney injury or a subject suspected of having acute kidney injury risk, applying the biological sample to an analytical device that is programmed to detect the concentration of at least two protein markers in the sample calculate the concentrations against a quantification standard, and transform the normalized concentrations, and calculate a score using an algorithm. The methods include optionally determining the status of at least one clinical variable or measurement, calculating a prognostic score using an algorithm based on the normalized, transformed concentrations of protein markers and optionally, the status of the clinical variable(s), classifying the prognostic score as a positive, intermediate, or negative result, and determining the risk of acute kidney injury in the subject as indicated by the prognostic score. The at least two protein markers are selected from Table 1. The optional clinical variable(s) or measurement(s) are selected from Table 2. The method includes repeating the steps as described herein using a biological sample from the same subject at a later timepoint, and comparing the scores to determine if there is a change in acute kidney injury risk in the subject.

In an aspect, provided herein are methods of administering a therapeutic intervention to a subject suspected of having acute kidney injury risk. The methods include (i) determining the subject's protein marker profile for a panel of at least two protein markers selected from Table 1; (ii) optionally, determining the status of at least one clinical variable or measurement for the subject, where the clinical variable or measurement is selected from Table 2; (iii) assigning a score to the subject based on the protein marker profile in (i) and optionally the clinical value status in (i); and (ii) administering to the subject a therapeutic intervention based on the positive, intermediate or negative score. Provided in the methods herein, the score is selected from positive, intermediate, and negative, and the score is algorithmically-derived based on the normalized, mathematically transformed concentrations of protein markers in the subject's sample and optionally, the status of at least one clinical variable or measurement.

In an aspect, provided herein are methods of detecting two or more protein markers in a subject having diabetes mellitus type 2 and/or that is suspected of having acute kidney injury risk. The methods include selecting a subject that has diabetes mellitus type 2 and/or that is suspected of having acute kidney injury risk, providing a biological sample from the subject, applying the biological sample to an analytical device, and detecting the concentration of at least two protein markers selected from Table 1. The optional clinical variable(s) or measurement(s) are selected from Table 2. The methods include detecting the blood urea nitrogen:creatinine ratio.

In an aspect, provided herein are methods of diagnosing risk of procedural acute kidney injury. The methods include providing a biological sample from a subject, applying the biological sample to an analytical device that is programmed to detect the concentration of at least two protein markers in the sample, normalize the concentrations against synthetic quantification standards, and transform the normalized concentrations into a score. The methods include optionally determining the status of at least one clinical variable, calculating a score based on the transformed, normalized concentrations of protein markers and optionally, the status of the clinical variable(s), classifying the score as a positive, intermediate, or negative result, and determining acute kidney injury in the subject as indicated by the score. The protein markers are selected from Table 1. The optional clinical variable(s) or measurement(s) are selected from Table 2.

In embodiments, a positive score indicates strong likelihood or presence of acute kidney injury. In embodiments, an intermediate score indicates a possible presence or likelihood of acute kidney injury. In embodiments, a negative score indicates absence or a weak likelihood of acute kidney injury.

### Embodiments

In certain specific embodiments, protein markers, optionally used in conjunction with clinical variables, can be used in methods for the prognosis of procedural acute kidney injury. In some embodiments, the protein markers are selected from CD5 antigen like, C reactive protein, Factor VII, kidney injury molecule 1, N terminal prohormone of brain natriuretic peptide, and osteopontin in conjunction with clinical measurement of blood urea nitrogen:creatinine ratio and the clinical variable of history of diabetes mellitus type 2. In some embodiments, the protein markers are CD5 antigen like, C reactive protein, Factor VII, and osteopontin in conjunction with clinical measurement of blood urea nitrogen:creatinine ratio and the clinical variable of history of diabetes mellitus type 2. In some embodiments, the protein markers are CD5 antigen like, C reactive protein, Factor VII, kidney injury molecule 1, and osteopontin in conjunction with clinical measurement of blood urea nitrogen:creatinine ratio and the clinical variable of history of diabetes mellitus type 2. In some embodiments, the protein markers are C reactive protein, kidney injury molecule 1 and osteopontin in conjunction with clinical measurement of blood urea nitrogen:creatinine ratio and the clinical variable of history of diabetes mellitus type 2. In some embodiments, the protein markers are C reactive protein and N-terminal prohormone of brain natriuretic peptide in conjunction with clinical measurement of blood urea nitrogen:creatinine ratio. In some embodiments, one or more (any combination) of the above-listed protein markers can be specifically excluded from any of the embodiments and aspects described herein.

### Assay

In embodiments, the biological sample includes whole blood, plasma, serum, urine, cerebral spinal fluid, biological fluid, and/or tissue samples. In some embodiments, the sample is whole blood. In some embodiments, the sample is plasma. In other embodiments, the sample is serum or urine.

Determining protein marker concentrations in a sample can be accomplished according to standard techniques known and available to the skilled artisan. In many instances, this will involve carrying out protein detection methods, which provide a quantitative measure of protein markers present in a biological sample.

In embodiments, target-binding agents that specifically bind to the protein markers described herein allow for a determination of the concentrations of the protein markers in a biological sample. Any of a variety of binding agents may be used including, for example, antibodies, polypeptides, sugars, aptamers, and nucleic acids.

In embodiments, the target-binding agent is an antibody or a fragment thereof that specifically binds to a protein marker as provided herein, and that is effective to determine the concentration of the protein marker to which it binds in a biological sample.

The term “specifically binds” or “binds specifically,” in the context of binding interactions between two molecules, refers to high avidity and/or high affinity binding of an antibody (or other binding agent) to a specific polypeptide subsequence or epitope of a protein marker. Antibody binding to an epitope on a specific protein marker sequence (also referred to herein as “an epitope”) is preferably stronger than binding of the same antibody to any other epitope, particularly those that may be present in molecules in association with, or in the same sample, as the specific protein marker of interest. Antibodies which bind specifically to a protein marker of interest may be capable of binding other polypeptides at a weak, yet detectable, level (e.g., 10% or less, 5% or less, 1% or less of the binding shown to the polypeptide of interest). Such weak binding, or background binding, is readily discernible from the specific antibody binding to the compound or polypeptide of interest, e.g. by use of appropriate controls. In general, antibodies used in compositions and methods described herein which bind to a specific protein marker with a binding affinity of 107 moles/L or more, preferably 108 moles/L or more are said to bind specifically to the specific protein marker.

In embodiments, the affinity of specific binding of an antibody or other binding agent to a protein marker is about 2 times greater than background binding, about 5 times greater than background binding, about 10 times greater than background binding, about 20 times greater than background binding, about 50 times greater than background binding, about 100 times greater than background binding, or about 1000 times greater than background binding, or more.

In embodiments, the affinity of specific binding of an antibody or other binding agent to a protein marker is between about 2 to about 1000 times greater than background binding, between about 2 to 500 times greater than background binding, between about 2 to about 100 times greater than background binding, between about 2 to about 50 times greater than background binding, between about 2 to about 20 times greater than background binding, between about 2 to about 10 times greater than background binding, or any intervening range of affinity.

In embodiments, the concentration of a protein marker is determined using an assay or format including, but not limited to, e.g., immunoassays, ELISA sandwich assays, lateral flow assays, flow cytometry, mass spectrometric detection, calorimetric assays, binding to a protein array (e.g., antibody array), single molecule detection methods, nanotechnology-based detection methods, or fluorescent activated cell sorting (FACS). In some embodiments, an approach involves the use of labeled affinity reagents (e.g., antibodies, small molecules, etc.) that recognize epitopes of one or more protein marker proteins in an immunoassay, antibody-labelled fluorescent bead array, antibody array, or FACS screen. As noted, any of a number of illustrative methods for producing, evaluating and/or using antibodies for detecting and quantifying the protein markers herein are well known and available in the art. It will also be understood that the protein detection and quantification in accordance with the methods described herein can be carried out in single assay format, multiplex format, or other known formats.

In embodiments, the concentration of a given protein is normalized to a quantification standard. In embodiments, the quantification standard is synthetic. A number of normalization methods are known in the art.

A number of suitable high-throughput multiplex formats exist for evaluating the disclosed protein markers. Typically, the term “high-throughput” refers to a format that performs a large number of assays per day, such as at least 100 assays, 1000 assays, up to as many as 10,000 assays or more per day. When enumerating assays, either the number of samples or the number of markers assayed can be considered.

In some embodiments, the samples are analyzed on an assay system or analytical device. For example, the assay system or analytical device may be a multiplex analyzer that simultaneously measures multiple analytes, e.g., proteins, in a single microplate well. The assay format may be receptor-ligand assays, immunoassays, and enzymatic assays. An example of such an analyzer is the Luminex® 100/200 system which is a combination of three xMAP® Technologies. The first is xMAP microspheres, a family of fluorescently dyed micron-sized polystyrene microspheres that act as both the identifier and the solid surface to build the assay. The second is a flow cytometry-based instrument, the Luminex® 100/200 analyzer, which integrates key xMAP® detection components, such as lasers, optics, fluidics, and high-speed digital signal processors. The third component is the xPONENT® software, which is designed for protocol-based data acquisition with robust data regression analysis.

By determining protein marker levels and optionally clinical variable status for a subject, a dataset may be generated and used (as further described herein) to classify the biological sample to one or more of risk stratification, prognosis, diagnosis, and monitoring of the cardiovascular status of the subject, and further assigning a likelihood of a positive, intermediate, or negative diagnosis, outcome, or one or more future changes in cardiovascular status to the subject to thereby establish a diagnosis and/or prognosis of cardiovascular disease and/or outcome, as described herein. The dataset may be obtained via automation or manual methods.

### Statistical Analysis

By analyzing combinations of protein markers and optional clinical variables as described herein, the methods described are capable of discriminating between different endpoints. The endpoints may include, for example, acute kidney injury risk. The identity of the markers and their corresponding features (e.g., concentration, quantitative levels) are used in developing and implementing an analytical process, or plurality of analytical processes, that discriminate between clinically relevant classes of patients.

Methods described herein may utilize machine learning. Machine learning is a field of statistics and computer science where algorithms generate models from data for the sake of prediction, regression, or classification. Machine learning algorithms generally require a set of “features”, which are the variables that are used to predict an “outcome” or “class”. In embodiments herein, the features are the normalized, log-transformed protein concentrations and the clinical factors, and the class or outcome is the medical outcome that we are trying to predict. The accuracy of learning models can be evaluated with many different metrics, depending on the type of class that the model is trying to predict (e.g., different metrics will be used for a binary outcome (e.g., “positive” vs. “negative”) than for a tertiary or continuous numeric outcome (the amount of obstruction present in a given artery). Machine learning gives computers the ability to learn without being explicitly programmed. Machine learning explores the study and construction of algorithms that can learn from and make predictions on data—such algorithms overcome following strictly static program instructions by making data-driven predictions or decisions through building a model from sample inputs. Machine learning is employed in a range of computing tasks where designing and programming explicit algorithms with good performance is difficult or infeasible. As a scientific endeavor, machine learning grew out of the quest for artificial intelligence (AI) and is considered a subset of artificial intelligence. Already in the early days of AI, some researchers were interested in having machines learn from data. They attempted to approach the problem with various symbolic methods, as well as what were then termed “neural networks”. Probabilistic reasoning was also employed, especially in automated medical diagnosis.

A protein marker and clinical variable dataset may be used in an analytic process for correlating the assay result(s) generated by the assay system and optionally the clinical variable status to the status of the subject, wherein said correlation step comprises correlating the assay result(s) to one or more of risk stratification, prognosis, diagnosis, classifying and monitoring of procedural acute kidney injury risk status of the subject, monitoring of procedural acute kidney injury risk effects of pharmacologic agents, identifying high risk patients for clinical trial enrollment, also referred to as clinical trial enrichment, or use as a companion diagnostic or complementary diagnostic for pharmacologic agents known of or suspected of causing kidney injury, wherein said correlating step comprises assigning a likelihood of a positive, intermediate, or negative diagnosis and/or prognosis, or one or more future changes in procedural acute kidney injury risk status to the subject based on the assay result(s).

A protein marker and clinical variable dataset may be used in an analytic process for generating a diagnostic and/or prognostic result or score. For example, an illustrative analytic process can comprise a linear model with one term for each component (protein level or clinical factor). The result of the model is a number that generates a diagnosis and/or prognosis. The model allows for the establishment of an algorithm for a particular protein marker and/or clinical variable dataset which is then used to generate a score. The result may also provide a multi-level or continuous score with a higher number representing a higher likelihood of disease or risk of event, a lower number representing a lower likelihood of disease or risk of event, and an intermediate number representing an intermediate likelihood of disease or risk of event.

The examples below illustrate how data analysis algorithms can be used to construct a number of such analytical processes. Each of the data analysis algorithms described in the examples uses features (e.g., normalized and transformed quantitative protein concentrations and/or clinical factors) of a subset of the markers identified herein across a training population. Specific data analysis algorithms for building an analytical process or plurality of analytical processes, that discriminate between subjects disclosed herein will be described in the subsections below. Once an analytical process has been built using these example data analysis algorithms or other techniques known in the art, the analytical process can be used to classify a test subject into one of the two or more phenotypic classes and/or predict survival/mortality or a severe medical event within a specified period of time after the blood test is obtained. This is accomplished by applying one or more analytical processes to one or more marker profile(s) obtained from the test subject. Such analytical processes, therefore, have enormous value as diagnostic or prognostic indicators.

In embodiments, the methods provide for normalization and transformation of the concentrations of a panel of protein markers, as described above, and subsequent use of an algorithm to convert the normalized, transformed concentration data into a score that may be used to determine whether a patient is diagnosed with acute kidney injury or has a prognosis of risk for acute kidney injury.

The data are processed prior to the analytical process. The data in each dataset are collected by measuring the values for each marker, usually in duplicate or triplicate or in multiple replicates. The data may be manipulated; for example, raw data may be transformed using standard curves, and the average of replicate measurements used to calculate the average and standard deviation for each patient. These values may be transformed before being used in the models, e.g., log-transformed, normalized to a standard scale, Winsorized, etc. The data is transformed via computer software and/or with instruction of an expert. This data can then be input into the analytical process with defined parameters.

The direct concentrations of the proteins (after log-transformation and normalization), the presence/absence of clinical factors represented in binary form (e.g., history of diabetes mellitus type 2, sex), and/or clinical factors in quantitative form (e.g., BMI, age, BUN:creatinine ratio) provide values that are plugged into the algorithmically-weighted diagnostic and/or prognostic model provided by the software, and the result is evaluated against one or more cutoffs to determine the diagnosis or prognosis.

The following are examples of the types of statistical analysis methods that are available to one of skill in the art to aid in the practice of the disclosed methods, panels, assays, and kits. The statistical analysis may be applied for one or both of the two following tasks: (1) these and other statistical methods may be used to identify preferred subsets of markers and other indices that will form a preferred dataset; (2) these and other statistical methods may be used to generate the analytical process that will be used with the dataset to generate the result. Several statistical methods presented herein or otherwise available in the art will perform both of these tasks and yield a model that is suitable for use as an analytical process for the practice of the methods disclosed herein.

Prior to analysis, the data is partitioned into a training set and a validation set. The training set is used to train, evaluate and build the final diagnostic or prognostic model. The validation set is not used at all during the training process, and is only used to validate final diagnostic or prognostic models.

The creation of training and validation sets can be done through random selection, or through chronological selection (i.e., where the training set is the first sequential set of patients, and the validation set is the second/final sequential set of patients). After these sets are determined, the balance of various outcomes (e.g., acute kidney injury risk, etc.) is considered to confirm that the outcomes of interest are properly represented in each data set.

In cases where sample sizes are small, the entire population of patients is used to train, evaluate, and develop a diagnostic or prognostic panel. All processes below, except when explicitly mentioned, involve the use of the entire population.

The features (e.g., proteins and/or clinical factors) of the diagnostic and/or prognostic models are selected for each outcome using a combination of analytic processes, including least angle regression (LARS; a procedure based on stepwise forward selection), shrinkage in statistical learning methods such as least absolute shrinkage and selection operator (LASSO), significance testing, and expert opinion.

The statistical learning method used to generate a result (classification, survival/mortality within a specified time, etc.) may be any type of process capable of providing a result useful for classifying a sample (e.g., a linear model, a probabilistic model, a decision tree algorithm, or a comparison of the obtained dataset with a reference dataset).

The diagnostic or prognostic signal in the features is evaluated with these statistical learning methods using a cross-validation procedure. For each cross-validation fold, the data (either the training set or all patients, depending on the sample size) is further split into training and validation sets (hereby called CV-training and CV-validation data sets).

For each fold of cross validation, the diagnostic or prognostic model is built using the CV-training data, and evaluated with the CV-validation data.

Models during the cross-validation process are evaluated with standard metrics of classification accuracy, e.g., the area under the ROC curve (AUC), sensitivity (Sn), specificity (Sp), positive predictive values (PPV), and negative predictive values (NPV).

Once a set of features (e.g., quantitative protein concentrations and optionally clinical factors) are selected to compose a final diagnostic or prognostic panel, a final predictive model is built using all of the training data.

Applying the patient data (e.g., quantitative protein concentrations and/or clinical factors) into the final predictive model yields a classification result. These results can be compared against a threshold for classifying a sample within a certain class (e.g., positive, intermediate, or negative diagnosis and/or prognosis, or a severity/likelihood score).

For small populations, a final model is created with the entire population, and then this model is evaluated again with the population to determine the in-sample diagnostic or prognostic results.

For populations of sufficient size to warrant separation into training and validation sets, final models are evaluated with the validation data set. To respect the authority of the validation data set, it is not used in an iterative way, to feed information back into the training process. It is only used as the final step of the analytic pipeline.

Models are evaluated with the entire population (for smaller populations) or with the validation data set (for populations of sufficient size to warrant separation into training and validation set), using metrics of diagnostic accuracy, including the AUC, sensitivity, specificity, positive predictive value and/or negative predictive value. Other metrics of accuracy, such as hazard ratio, relative risk, and net reclassification index are considered separately for models of interest.

This final model or a model optimized for a particular protein marker platform, when used in a clinical setting, may be implemented as a software system, running directly on the assay hardware platform or on an independent system. The model may receive protein level or concentration data directly from the assay platform or other means of data transfer, and patient clinical data may be received via electronic, manual, or other query of patient medical records or through interactive input with the operator. This patient data may be processed and run through the final model, which will provide a result to clinicians, medical staff, and/or researchers for purposes of decision support.

In embodiments, the protein markers and/or clinical variables include those listed in Table 1. In embodiments, protein markers include those listed in Table 3, particularly those that are associated with a p-value of less than 0.1, less than 0.05, less than 0.01 or less than 0.001.

In some embodiments, at least 2, at least 3 or at least 4 protein markers are used in the methods provided herein. In other embodiments, the number of protein markers employed can vary, and may include at least 5, 6, 7, 8, 9, 10, or more. In still other embodiments, the number of protein markers can include at least 15, 20, 25 or 50, or more.

In embodiments, the methods provided herein include measuring the concentrations of at least two protein markers selected from Table 1. In embodiments, the methods provided herein include measuring the concentrations of at two protein markers selected from Table 1. In embodiments, the methods provided herein include measuring the concentrations of three protein markers selected from Table 1. In embodiments, the methods provided herein include measuring the concentrations of four protein markers selected from Table 1. In embodiments, the methods provided herein include measuring the concentrations of five protein markers selected from Table 1. In embodiments, the methods provided herein include measuring the concentrations of six protein markers selected from Table 1. Such determination can be made by standard methods known in the art and described herein. In embodiments, measurement of the concentrations of the protein markers selected from Table 1 determines a subject's protein profile.

In embodiments, the analytical device for measuring the concentrations of protein markers is an immunoassay device. The device may be configured with software controls and analytical programs capable of mathematical computations such as normalizing detected protein marker concentrations against a quantification standard. The quantification standard may be part of the protein detection assay or may be separately contained. The software controls and analytical programs may be further capable of receiving clinical variables entered as a mathematical factor and log-transforming the normalized concentrations into a value that is then converted into a score based on pre-entered algorithms and models to accept the protein marker concentrations and the optional clinical variable(s). The mathematical log-transformations and use of an algorithm to generate a diagnostic and/or prognostic score can be accomplished within the analytical device, computer, in a cloud computing setting or the like.

In embodiments, the status of at least one clinical variable or measurement selected from Table 2 is determined. In embodiments, the methods provided herein include determining the status of one clinical variable selected from Table 2. In embodiments, the methods provided herein include determining the status of two clinical variables selected from Table 2. In embodiments, the methods provided herein include determining the status of three clinical variable selected from Table 2. In embodiments, the methods provided herein include determining the status of four clinical variable selected from Table 2. Such determination can be made by standard methods known in the art such as medical history review, electronic health records (EHR) or other information system, or clinical lab tests.

In embodiments, assigning a score to the subject based on the protein marker profile and optionally the clinical value status can be accomplished using a device configured with software controls and analytical programs capable of mathematical computations as described above. The score may be classified as a positive, intermediate, or negative diagnostic result. The score may be classified as a positive, intermediate, or negative prognostic result.

### Scoring and Treatments

In some embodiments, the diagnostic or prognostic calculations will result in a numeric or categorical score that relates the patient's level of likelihood of AKI risk, e.g., including but not limited to positive predictive value (PPV), negative predictive value (NPV), sensitivity (Sn), or specificity (Sp) or the risk of a cardiovascular event or outcome, such as AKI risk, occurring within the specified period. The number of levels used by the diagnostic or prognostic model may be as few as two (“positive” vs. “negative”) or as many as deemed clinically relevant, e.g., a prognostic model for AKI may result a five-level score, where a higher score indicates a higher likelihood of AKI risk. Specifically, a score of 1 indicates a strong degree of confidence in a low likelihood of AKI risk or a negative result (determined by the test's NPV or Sn), a score of 5 indicates a strong degree of confidence in a high likelihood of AKI risk or a positive result (determined by the test's PPV or Sp), and a score of 3 indicates an intermediate or moderate likelihood for AKI risk.

In embodiments, the methods provided herein further include treating the subject based on a positive, intermediate or negative prognostic score for acute kidney injury risk. Treating the subject includes providing a therapeutic regimen. The therapeutic regimen may include administration of therapeutic drugs, further diagnostic testing, lifestyle modification, surgical intervention and the like. In embodiments, a positive prognostic score in the subject facilitates a determination by a medical practitioner of the need for one or more interventions selected from ultrasound, administration of pharmacological agents, hydration, delaying a cardiac catheterization or other dye-based procedure and avoidance of any drug or procedure with a known kidney risk. In embodiments, a negative prognostic score in the subject facilitates a determination by a medical practitioner of the need for one or more interventions selected from ongoing monitoring and management of peripheral and coronary risk factors, and proceeding with a cardiac catheterization or other dye-based procedure. In embodiments, an intermediate diagnostic score in the subject facilitates a determination by a medical practitioner of the need for one or more interventions selected from further testing, proceeding with a cardiac catheterization or other dye-based procedure whereby dye usage is strictly limited, more frequent monitoring for risk factors and lifestyle modifications.

### Panels, Assays, and Kits

Provided herein are panels, assays, and kits comprising target-binding agents that bind at least 2, at least 3, at least 4 or greater than 4 protein markers and optionally clinical variable(s), in order to aid or facilitate a diagnostic or prognostic finding according to the present disclosure. For example, in some embodiments, a diagnostic or prognostic panel or kit comprises one or a plurality of protein markers set out in Table 1 and optionally one or a plurality of applicable clinical variables set out in Table 2.

It will be understood that, in many embodiments, the panels, assays, and kits described herein comprise antibodies, binding fragments thereof and/or other types of target-binding agents which are specific for the protein markers of Table 1, and which are useful for determining the concentrations of the corresponding protein marker in a biological sample according to the methods describe herein. Accordingly, in each description herein of a panel, assay, or kit comprising one or a plurality of protein markers, it will be understood that the very same panel, assay, or kit can advantageously comprise, in addition or instead, one or a plurality of antibodies, binding fragments thereof or other types of binding agents such as aptamers, which are specific for the protein markers of Table 1. Of course, the panels, assays, and kits can further comprise, include or recommend a determination of one or a plurality of applicable clinical variables as set out in Table 2.

In certain specific embodiments, the protein markers and/or clinical variables used in in conjunction with a panel, assay, or kit include those listed in Table 1 and Table 2 respectively, particularly those which are associated with a p-value of less than 0.1, less than 0.05, less than 0.01 or less than 0.001.

In some embodiments, panels, assays, and kits may comprise at least 2, at least 3, at least 4, or at least 5 target-binding agents specific for protein markers as described herein. In embodiments, panels, assays, and kits may comprise target-binding agents for two protein markers. In embodiments, panels, assays, and kits may comprise target-binding agents for three protein markers. In embodiments, panels, assays, and kits may comprise target-binding agents for four protein markers. In embodiments, panels, assays, and kits may comprise target-binding agents for five protein markers. In other embodiments, the number of protein markers employed can include at least 6, 7, 8, 9 or 10 or more. In still other embodiments, the number of protein markers employed can include at least 15, 20, 25 or 50, or more.

As described herein, panels, assays, and kits of the present disclosure can be used for identifying the presence of adverse procedural outcomes in a subject, particularly the presence of acute kidney injury and/or for predicting adverse procedural outcomes such as risk of acute kidney injury. In some embodiments, a prognostic panel, assay, or kit identifies in a subject the risk of procedural acute kidney injury.

In other embodiments, a prognostic, companion diagnostic, and/or complementary diagnostic panel, assay, or kit is used to predict the risk of acute kidney injury or event within one week, within 2 weeks, within 3 weeks, within a month, within one year, about 1 year, about 2 years, about 3 years, about 4 years, about 5 years, or more from the date on which the sample is drawn. Time endpoints are defined as from sample draw and include less than one year, one year, and greater than one year. Less than or within one year may be any time from time of sample draw up to and including 365 days. For example, the panel results may predict the risk of acute kidney injury risk from time of sample draw to 1 day, to 2 days, to 3 days, to 4 days, to 5 day, to 6 days, to 7 days, to 8 days, to 9 days, to 10 days, to 20 days, to 30 days, to 60 days, to 90 days, to 120 days, to 150 days, to 180 days, to 210 days, to 240 days, to 270 days, to 300 days, to 330 days, to 360 days, to 365 days. In yet other embodiments, time endpoints are defined as 3 days post sample draw to 30 days, 3 days to 60 days, 3 days to 90 days, 3 days to 120 days, 3 days to 150 days, 3 days to 180 days, 3 days to 210 days, 3 days to 240 days, 3 days to 270 days, 3 days to 300 days, 3 days to 330 days, 3 days to 360 days, to 3 days 365 days. Suitable time frames include any value or subrange within the recited range, including endpoints.

In specific embodiments, panels, assays, and kits for the prognosis of risk of procedural acute kidney injury (AKI) and/or monitoring AKI progression comprise at least 2, at least 3, at least 4, at least 5 or greater than five protein markers, or antibodies, binding fragments thereof or other types of binding agents, which are specific for the protein markers, where the protein markers are selected from CD5 antigen-like, C reactive protein, Factor VII, kidney injury molecule 1, N-terminal prohormone of brain natriuretic peptide, and osteopontin. In some embodiments, at least one clinical variable or measurement described herein is used in conjunction with the protein marker concentrations determined. In other embodiments, the clinical variables/measurements are blood urea nitrogen:creatinine ratio and history of diabetes mellitus type 2. In yet other embodiments, the clinical variable/measurement is blood urea nitrogen creatinine ratio.

In specific embodiments, a panel, assay, or kit for the prognosis of acute kidney injury risk following cardiac procedures or interventions in a patient comprises the protein marker for C-reactive protein. In specific embodiments, a panel, assay, or kit for the prognosis of acute kidney injury risk following cardiac procedures or interventions in a patient comprises the protein marker for CD5 antigen-like. In specific embodiments, a panel, assay, or kit for the prognosis of acute kidney injury risk following cardiac procedures or interventions in a patient comprises the clinical measurement of blood urea nitrogen:creatinine ratio. In specific embodiments, a panel, assay, or kit for the prognosis of acute kidney injury risk following cardiac procedures or interventions in a patient comprises the clinical variable of history of diabetes type 2. In specific embodiments, a panel, assay, or kit for the prognosis of acute kidney injury risk following cardiac procedures or interventions in a patient comprises the protein marker for C-reactive protein and clinical measurement of blood urea nitrogen:creatinine ratio. In specific embodiments, a panel, assay, or kit for the prognosis of acute kidney injury risk following cardiac procedures or interventions in a patient comprises the protein marker for C-reactive protein and the clinical variable of history of diabetes type 2. In specific embodiments, a panel, assay, or kit for the prognosis of acute kidney injury risk following cardiac procedures or interventions in a patient comprises the protein marker for CD5 antigen-like and clinical measurement of blood urea nitrogen:creatinine ratio. In specific embodiments, a panel, assay, or kit for the prognosis of acute kidney injury risk following cardiac procedures or interventions in a patient comprises the protein marker for CD5 antigen-like and the clinical variable of history of diabetes type 2.

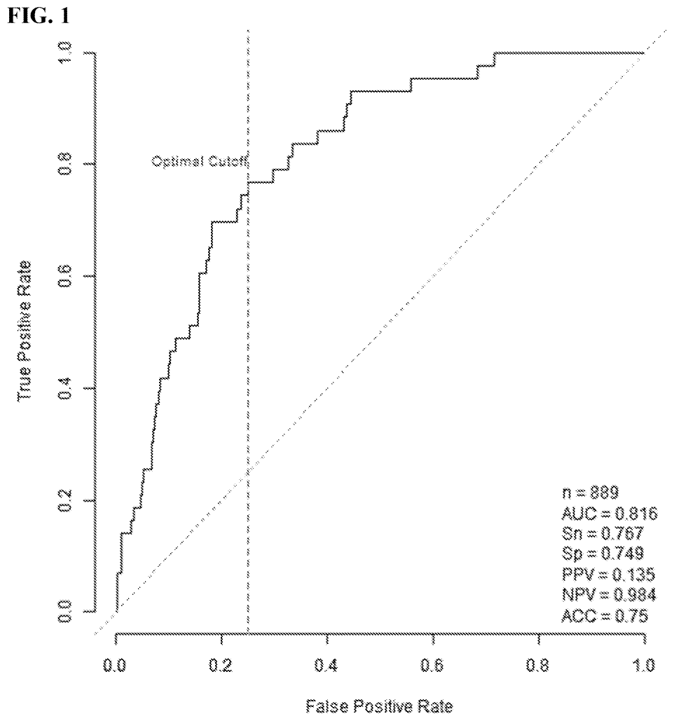

In specific embodiments, a panel, assay, or kit for the prognosis of acute kidney injury risk following cardiac procedures or interventions in a patient comprises protein markers for C-reactive protein, CD5 antigen-like, Factor VII, and osteopontin and the clinical variable/measurement of blood urea nitrogen:creatinine ratio and history of diabetes type 2. This combination of protein markers and clinical variables is represented by panel AKI 026e in Table 6, Example 1, and FIG. 1.

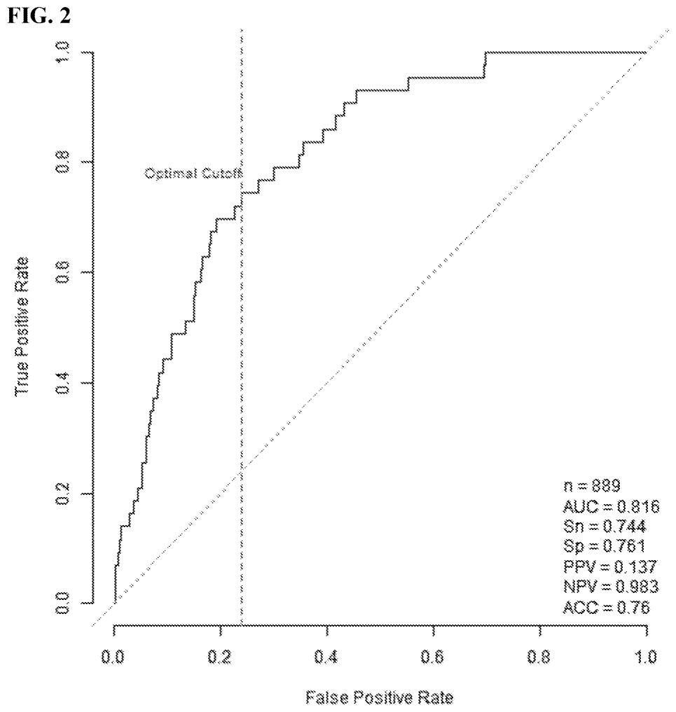

In specific embodiments, a panel, assay, or kit for the diagnosis of acute kidney injury following cardiac procedures or interventions in a patient comprises protein markers for C-reactive protein, CD5 antigen-like, Factor VII, kidney injury molecule 1, and osteopontin and the clinical variable/measurement blood urea nitrogen:creatinine ratio and history of diabetes type 2. This combination of protein markers and clinical variable is represented by panel AKI 027e in Table 6, Example 3, and FIG. 2.

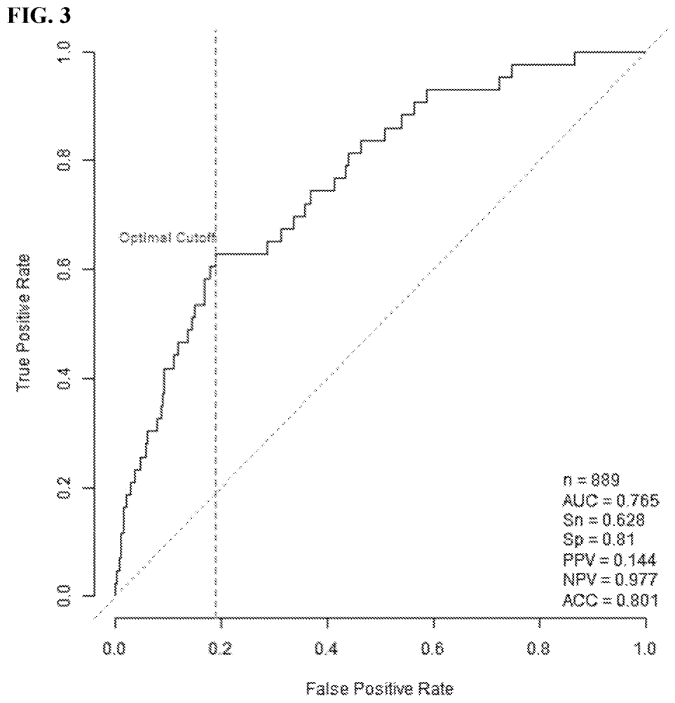

In specific embodiments, a panel, assay, or kit for the diagnosis of acute kidney injury following cardiac procedures or interventions in a patient comprises protein markers for C-reactive protein, kidney injury molecule 1, and osteopontin and the clinical variable/measurement of blood urea nitrogen:creatinine ratio and history of diabetes type 2. This combination of protein markers and clinical variable is represented by panel AKI 032e in Table 6, Example 4, and FIG. 3.

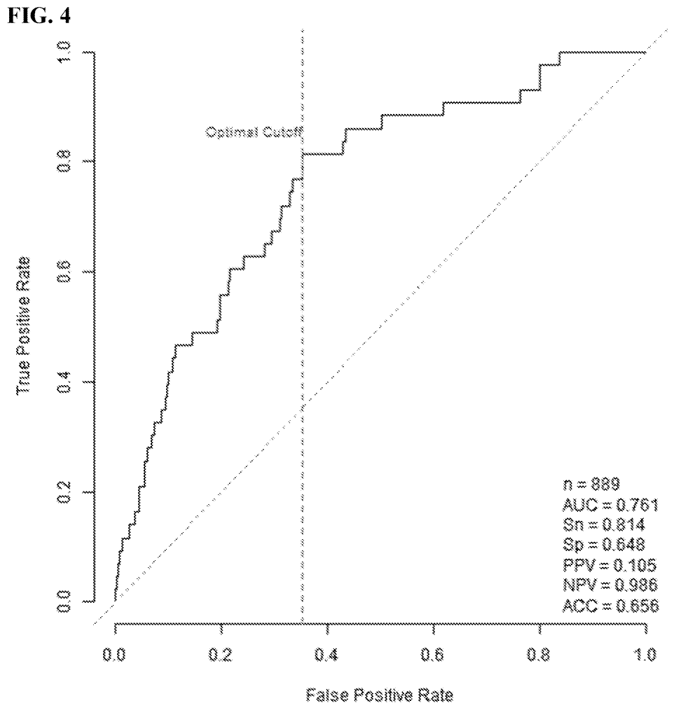

In specific embodiments, a panel, assay, or kit for the diagnosis of acute kidney injury following cardiac procedures or interventions in a patient comprises protein markers for C-reactive protein and N-terminal prohormone of brain natriuretic peptide and the clinical variable/measurement of blood urea nitrogen:creatinine ratio. This combination of protein markers and clinical variable is represented by panel AKI 052e in Table 6, Example 5, and FIG. 4.

In certain embodiments, a panel, assay, or kit comprises at least 2, at least 3, at least 4, at least 5 or greater than 5 antibodies or binding fragments thereof, or other types of binding agents, where the antibodies, binding fragments or other binding agents are specific for a protein marker of Table 1.

It will be understood that the panels, assays, and kits of the present disclosure may further comprise virtually any other compounds, compositions, components, instructions, or the like, that may be necessary or desired in facilitating a determination of a diagnosis or prognosis according to the present disclosure. These may include instructions for using the panel, assay, or kit, instructions for making a diagnostic or prognostic determination (e.g., by calculating a diagnostic or prognostic score), instructions or other recommendations for a medical practitioner in relation to preferred or desired modes of therapeutic or diagnostic intervention in the subject in light of the diagnostic or prognostic determination, and/or monitoring therapeutic effects and the like.

In some embodiments, the panels, assays, and kits as described herein will facilitate detection of the protein markers discussed herein. Means for measuring such blood, plasma and/or serum concentrations are known in the art, and include, for example, the use of an immunoassay.

In addition to the methods described above, any method known in the art for quantitatively measuring concentrations of protein in a sample, e.g., non-antibody-based methods can be used in the methods and kits described herein. For example, mass spectrometry-based (such as, for example, Multiple Reaction Monitoring (MRM) mass spectrometry) or HPLC-based methods can be used. Methods of protein quantification [described in 31-36].

Additionally, technologies such as those used in the field of proteomics and other areas may also be embodied in methods, kits and other aspects described herein. Such technologies include, for example, the use of micro- and nano-fluidic chips, biosensors and other technologies as described, for example, in United States Patent Application Nos. US2008/0202927; US2014/0256573; US2016/0153980; WO2016/001795; US2008/0185295; US2010/0047901; US2010/0231242; US2011/0154648; US2013/0306491; US2010/0329929; US2013/0261009; [37-47].

### EXAMPLES

### Example 1: a Clinical and Protein Marker Scoring System Prognose Acute Kidney Injury (AKI) Risk, Panel AKI 026e

Standard measures of kidney function are only modestly useful for accurate prediction of risk for acute kidney injury (AKI) following coronary angiography. Using Luminex xMAP technology, 112 biomarkers in blood were measured from 889 patients prior to undergoing coronary angiography. Procedural AKI was defined as an abrupt reduction in kidney function with an absolute increase in serum creatinine of more than or equal to 0.3 mg/dL, a percentage increase in serum creatinine of >50%, or a reduction in urine output (documented oliguria of <0.5 mL/kg per hour for >6 hours) within 7 days after contrast exposure. Clinical and biomarker predictors of AKI risk were identified using machine learning and a final prognostic model was developed with least absolute shrinkage and selection operator (LASSO). Forty-three (4.8%) patients developed procedural AKI.

TABLE 3
is a list of Protein Markers tested.

| | With Procedural | | Without | | |
|---|---|---|---|---|---|
| Protein Marker | AKI | | Procedural AKI | | p |
| Adiponectin (ug/mL) | 4.5 | (2.6, 6.9) | 3.7 | (2.4, 5.6) | 0.16 |
| Alpha-1-Antitrypsin (AAT) (mg/mL) | 2.2 | (1.8, 2.6) | 1.8 | (1.5, 2.1) | <0.001 |
| Alpha-2-Macroglobulin (A2Macro) (mg/mL) | 2.2 | (1.7, 2.7) | 1.9 | (1.5, 2.3) | 0.05 |
| Angiopoietin-1 (ANG-1) (ng/mL) | 7.5 | (5.4, 12) | 6.8 | (4.9, 10) | 0.24 |
| Angiotensin-Converting Enzyme (ACE) (ng/mL) | 82 | (58.5, 104.5) | 79 | (61.3, 104.8) | 1.00 |
| Apolipoprotein(a) (Lp(a)) (ug/mL) | 197 | (62, 443) | 202.5 | (69.3, 493.8) | 0.64 |
| Apolipoprotein A-I (Apo A-I) (mg/mL) | 1.8 | (1.5, 2.2) | 1.8 | (1.5, 2.2) | 0.95 |
| Apolipoprotein A-II (Apo A-II) (ng/mL) | 297 | (269, 354) | 313.5 | (252, 385) | 0.35 |
| Apolipoprotein B (Apo B) (ug/mL) | 1190 | (899, 1645) | 1410 | (1090, 1860) | 0.005 |
| Apolipoprotein C-I (Apo C-I) (ng/mL) | 307 | (274.5, 361) | 317.5 | (260, 380) | 0.93 |
| Apolipoprotein C-III (Apo C-III) (ug/mL) | 211 | (173.5, 255.5) | 215 | (159, 268.8) | 0.87 |
| Apolipoprotein H (Apo H) (ug/mL) | 312 | (266.5, 368) | 331 | (271.3, 389.8) | 0.34 |
| Beta-2-Microglobulin (B2M) (ug/mL) | 2.1 | (1.8, 3) | 1.7 | (1.4, 2.3) | <0.001 |
| Brain-Derived Neurotrophic Factor (BDNF) | 2.6 | (1.3, 4.2) | 2.3 | (1, 4.7) | 0.43 |
| (ng/mL) | | | | | |
| C-Reactive Protein (CRP) (ug/mL) | 8.8 | (3.8, 22.5) | 3.5 | (1.5, 9.1) | <0.001 |
| Calbindin (ng/mL) | 8 | (8, 20) | 8 | (8, 8) | p = 0.01 |
| Carbonic anhydrase 9 (CA-9) (ng/mL) | 0.2 | (0.1, 0.3) | 0.14 | (0.1, 0.2) | 0.11 |
| Carcinoembryonic antigen-related cell adhesion | 25 | (22, 30.5) | 23 | (20, 27) | 0.07 |
| molecule 1 (CEACAM1) (ng/mL) | | | | | |
| CD5 Antigen-like (CD5L) (ng/mL) | 3600 | (2695, 5370) | 3755 | (2860, 5097.5) | 0.77 |
| Cystatin (mg/L) | 0.945 | (0.76, 1.088) | 0.79 | (0.68, 0.97 | P = <0.001 |
| Decorin (ng/mL) | 3.3 | (2.2, 4.4) | 2.3 | (1.9, 3.4) | 0.004 |
| E-Selectin (ng/mL) | 4.8 | (3.3, 6.1) | 5.2 | (3.7, 7) | 0.21 |
| EN-RAGE (ng/mL) | 39 | (18, 57) | 27 | (17, 48) | 0.09 |
| Eotaxin-1 (pg/mL) | 98 | (65.3, 154.5) | 95.5 | (42.5, 141) | 0.24 |
| Factor VII (ng/mL) | 350 | (290.5, 523) | 468 | (360, 588.8) | 0.005 |
| Fatty Acid-Binding Protein, heart (FABP, heart) | 4.6 | (4.6, 10.4) | 4.6 | (4.6, 4.6) | <0.001 |
| (ng/mL) | | | | | |
| Ferritin (FRTN) (ng/mL) | 139 | (70.5, 204) | 134 | (72.3, 232) | 0.96 |
| Fetuin-A (ug/mL) | 568 | (483, 777.5) | 698 | (588.3, 829) | 0.003 |
| Fibrinogen (mg/mL) | 5.2 | (4.2, 6.1) | 4.4 | (3.6, 5.4) | 0.002 |
| Follicle-Stimulating Hormone (FSH) (mIU/mL) | 7.2 | (3.7, 38) | 6.8 | (3.7, 28) | 0.72 |
| Glucagon-like Peptide 1, total (GLP-1 total) | 3.5 | (3.5, 3.5) | 3.5 | (3.5, 3.5) | 0.44 |
| (pg/mL) | | | | | |
| Granulocyte-Macrophage Colony-Stimulating | 10.5 | (10.5, 10.5) | 10.5 | (10.5, 10.5) | 0.75 |
| Factor (GM-CSF) (pg/mL) | | | | | |
| Growth Hormone (GH) (ng/mL) | 0.5 | (0.2, 1.4) | 0.3 | (0.1, 0.9) | 0.07 |
| Haptoglobin (mg/mL) | 1.5 | (0.9, 2.3) | 1.2 | (0.6, 1.9) | 0.05 |
| Immunoglobulin A (IgA) (mg/mL) | 2.6 | (1.8, 3.7) | 2.4 | (1.6, 3.4) | 0.27 |
| Immunoglobulin M (IgM) (mg/mL) | 1.2 | (0.9, 1.6) | 1.4 | (0.9, 2.1) | 0.13 |
| Insulin (uIU/mL) | 1.1 | (0.3, 2.3) | 0.8 | (0.1, 2.1) | 0.30 |
| Intercellular Adhesion Molecule 1 (ICAM-1) | 112 | (86.5, 132.5) | 104 | (85, 130) | 0.45 |
| (ng/mL) | | | | | |
| Interferon gamma (IFN-gamma) (pg/mL) | 1.3 | (1.3, 1.3) | 1.3 | (1.3, 1.3) | 0.003 |
| Interferon gamma Induced Protein 10 (IP-10) | 311 | (256, 466) | 304 | (233, 402.8) | 0.20 |
| (pg/mL) | | | | | |
| Interleukin-1 alpha (IL-1 alpha) (ng/mL) | 0.001 | (0.001, 0.001) | 0.001 | (0.001, 0.001) | 0.31 |
| Interleukin-1 beta (IL-1 beta) (pg/mL) | 3.3 | (3.3, 8.5) | 6.6 | (3.3, 8.4) | 0.84 |
| Interleukin-1 receptor antagonist (IL-1ra) (pg/mL) | 118 | (68.5, 162) | 114 | (87.3, 144) | 0.90 |
| Interleukin-2 (IL-2) (pg/mL) | 20.5 | (20.5, 20.5) | 20.5 | (20.5, 20.5) | 0.75 |
| Interleukin-3 (IL-3) (ng/mL) | 0.003 | (0.003, 0.003) | 0.003 | (0.003, 0.003) | 0.75 |
| Interleukin-4 (IL-4) (pg/mL) | 17.5 | (17.5, 17.5) | 17.5 | (17.5, 17.5) | 0.75 |
| Interleukin-5 (IL-5) (pg/mL) | 2.4 | (2.4, 2.4) | 2.4 | (2.4, 2.4) | 0.12 |
| Interleukin-6 (IL-6) (pg/mL) | 2.3 | (2.3, 2.3) | 2.3 | (2.3, 2.3) | 0.05 |
| Interleukin-6 receptor (IL-6r) (ng/mL) | 25 | (18.5, 31) | 24 | (19, 29) | 0.76 |
| Interleukin-7 (IL-7) (pg/mL) | 16 | (16, 16) | 16 | (16, 16) | 0.75 |
| Interleukin-8 (IL-8) (pg/mL) | 8.2 | (6.15, 13.5) | 6.4 | (4.4, 9.8) | 0.003 |
| Interleukin-10 (IL-10) (pg/mL) | 3.4 | (3.4, 3.4) | 3.4 | (3.4, 3.4) | 0.74 |
| Interleukin-12 Subunit p40 (IL-12p40) (ng/mL) | 0.6 | (0.4, 0.7) | 0.6 | (0.5, 0.7) | 0.45 |
| Interleukin-12 Subunit p70 (IL-12p70) (pg/mL) | 25 | (25, 25) | 25 | (25, 25) | 0.003 |
| Interleukin-15 (IL-15) (ng/mL) | 0.5 | (0.2, 0.7) | 0.6 | (0.5, 0.7) | 0.52 |
| Interleukin-17 (IL-17) (pg/mL) | 1.5 | (1.5, 1.5) | 1.5 | (1.5, 1.5) | 0.26 |
| Interleukin-18 (IL-18) (pg/mL) | 191 | (140, 264) | 200 | (149, 268) | 0.90 |
| Interleukin-18-binding protein (IL-18bp) (ng/mL) | 11 | (8.8, 17) | 9.2 | (7.1, 12) | 0.003 |
| Interleukin-23 (IL-23) (ng/mL) | 2.1 | (1.7, 3.1) | 2.5 | (2, 3.2) | 0.23 |
| Kidney Injury Molecule-1 (KIM-1) (ng/mL) | 0.05 | (0.03, 0.1) | 0.04 | (0.01, 0.06) | 0.004 |
| Lectin-Like Oxidized LDL Receptor 1 (LOX-1) | 0.3 | (0.3, 0.7) | 0.3 | (0.3, 0.3) | 0.04 |
| (ng/mL) | | | | | |
| Leptin (ng/mL) | 8.5 | (5.3, 20.5) | 8.9 | (4.5, 20) | 0.94 |
| Lipoprotein (a) (Lp(a)) (ug/mL) | 197 | (62, 443) | 202.5 | (69.3, 493.8) | 0.64 |
| Luteinizing Hormone (LH) (mIU/mL) | 5.7 | (3.1, 11) | 4.8 | (3.3, 9.8) | 0.52 |
| Macrophage Colony-Stimulating Factor 1 (M-CSF) | 0.7 | (0.4, 1.3) | 0.4 | (0.2, 0.6) | <0.001 |
| (ng/mL) | | | | | |
| Macrophage Inflammatory Protein-1 alpha (MIP-1 | 14.5 | (14.5, 37.5) | 14.5 | (14.5, 35) | 0.89 |
| alpha) (pg/mL) | | | | | |
| Macrophage Inflammatory Protein-1 beta (MIP-1 | 278 | (225.5, 391) | 268 | (195, 361) | 0.16 |
| beta) (pg/mL) | | | | | |
| Macrophage Inflammatory Protein-3 alpha (MIP-3 | 10 | (10, 29.5) | 10 | (10, 26) | 0.41 |
| alpha) (pg/mL) | | | | | |
| Matrix Metalloproteinase-1 (MMP-1) (ng/mL) | 0.3 | (0.3, 0.3) | 0.3 | (0.3, 0.3) | 0.78 |
| Matrix Metalloproteinase-2 (MMP-2) (ng/mL) | 1490 | (1300, 1825) | 1310 | (1110, 1610) | 0.002 |
| Matrix Metalloproteinase-3 (MMP-3) (ng/mL) | 7.5 | (5.7, 14.5) | 6.6 | (4.7, 9.8) | 0.06 |
| Matrix Metalloproteinase-7 (MMP-7) (ng/mL) | 0.4 | (0.2, 0.6) | 0.4 | (0.2, 0.5) | 0.83 |
| Matrix Metalloproteinase-9 (MMP-9) (ng/mL) | 126 | (92.5, 181.5) | 120 | (86, 170) | 0.46 |
| Matrix Metalloproteinase-9, total (MMP-9, total) | 626 | (430.5, 916) | 545 | (399.3, 775) | 0.14 |
| (ng/mL) | | | | | |
| Matrix Metalloproteinase-10 (MMP-10) (ng/mL) | 0.1 | (0.1, 0.1) | 0.1 | (0.1, 0.1) | 0.58 |
| Midkine (ng/mL) | 18 | (11, 27) | 14 | (9.9, 19) | 0.01 |
| Monocyte Chemotactic Protein 1 (MCP-1) | 127 | (88, 161) | 110 | (76, 161) | 0.23 |
| (pg/mL) | | | | | |
| Monocyte Chemotactic Protein 2 (MCP-2) | 26 | (17, 34) | 23 | (18, 29) | 0.26 |
| (pg/mL) | | | | | |
| Monocyte Chemotactic Protein 4 (MCP-4) | 2200 | (1650, 3260) | 2280 | (1620, 3397.5) | 0.93 |
| (pg/mL) | | | | | |
| Monokine Induced by Gamma Interferon (MIG) | 1230 | (829, 1830) | 915.5 | (578, 1607.5) | 0.04 |
| (pg/mL) | | | | | |
| Myeloid Progenitor Inhibitory Factor 1 (MPIF-1) | 1.4 | (1.2, 2) | 1.2 | (0.95, 1.5) | 0.008 |
| (ng/mL) | | | | | |
| Myeloperoxidase (pmol/L) | 462 | (333, 794.5) | 423 | (320, 592.75) | 0.125 |
| Myoglobin (ng/mL) | 35 | (24, 56) | 32 | (22, 46) | 0.11 |
| N-terminal pro B-type natriuretic peptide (NT | 4490 | (1780, 15975) | 1445 | (516.8, 3762.5) | <0.001 |
| proBNP) (pg/mL) | | | | | |
| Osteopontin (ng/mL) | 43 | (31.5, 66) | 27 | (20, 41) | <0.001 |
| Pancreatic Polypeptide (PPP) (pg/mL) | 115 | (63, 201) | 84 | (48, 158) | 0.05 |
| Plasminogen Activator Inhibitor 1 (PAI-1) (ng/mL) | 43 | (27.5, 76.5) | 46 | (27, 72.8) | 0.87 |
| Platelet endothelial cell adhesion molecule | 55 | (46, 68.5) | 54 | (45, 63) | p = 0.44 |
| (PECAM-1) (ng/mL) | | | | | |
| Prolactin (PRL) (ng/mL) | 8.6 | (5.9, 13) | 8.1 | (5.5, 12.8) | 0.33 |
| Pulmonary and Activation-Regulated Chemokine | 114 | (79, 173) | 100 | (74.3, 137) | 0.16 |
| (PARC) (ng/mL) | | | | | |
| Pulmonary surfactant-associated protein D (SP-D) | 6.8 | (4.6, 9.9) | 5.1 | (3.3, 8.3) | 0.02 |
| (ng/mL) | | | | | |
| Resistin (ng/mL) | 2.8 | (2.0, 3.8) | 2.4 | (1.8, 3.4) | 0.13 |
| Serotransferrin (Transferrin) (mg/dl) | 276 | (232.5, 308.5) | 272 | (234, 315) | 0.98 |
| Serum Amyloid P-Component (SAP) (ug/mL) | 12 | (9, 15.5) | 13 | (10, 16) | 0.21 |
| Stem Cell Factor (SCF) (pg/mL) | 400 | (309, 502) | 362 | (279, 449.8) | 0.09 |
| T-Cell-Specific Protein RANTES (RANTES) | 11 | (6.2, 18.5) | 8.5 | (3.9, 18) | 0.17 |
| (ng/mL) | | | | | |
| Tamm-Horsfall Urinary Glycoprotein (THP) | 0.03 | (0.02, 0.04) | 0.03 | (0.02, 0.04) | 0.02 |
| (ug/mL) | | | | | |
| Thrombomodulin (TM) (ng/mL) | 3.8 | (3.2, 5.05) | 3.8 | (3.1, 4.6) | 0.35 |
| Thrombospondin-1 (ng/mL) | 4450 | (3050, 7595) | 4625 | (2160, 7622.5) | 0.52 |
| Thyroid-Stimulating Hormone (TSH) (uIU/mL) | 1.3 | (0.7, 2.2) | 1.2 | (0.8, 1.9) | 0.79 |
| Thyroxine-Binding Globulin (TBG) (ug/mL) | 35 | (31, 42.5) | 38 | (31, 45) | 0.12 |
| Tissue Inhibitor of Metalloproteinases 1 (TIMP-1) | 92 | (72, 117.5) | 72 | (59, 90) | <0.001 |
| (ng/mL) | | | | | |
| Transthyretin (TTR) (mg/dl) | 21 | (18, 25.5) | 26 | (21, 30) | 0.002 |
| Troponin (ng/L) | 25.95 | (8.525, 195) | 8.05 | (3.6, 29.5) | <0.001 |
| Tumor Necrosis Factor alpha (TNF-alpha) (pg/mL) | 6.5 | (6.5, 6.5) | 6.5 | (6.5, 6.5) | <0.001 |
| Tumor Necrosis Factor beta (TNF-beta) (pg/mL) | 20 | (20, 20) | 20 | (20, 20) | 1.00 |
| Tumor necrosis factor receptor 2 (TNFR2) (ng/mL) | 8.1 | (5.75, 11) | 6.3 | (4.8, 8.7) | 0.003 |
| Vascular Cell Adhesion Molecule-1 (VCAM-1) | 628 | (488.5, 843) | 563.5 | (456, 706) | 0.03 |
| (ng/mL) | | | | | |
| Vascular Endothelial Growth Factor (VEGF) | 86 | (70.5, 145) | 98 | (68, 135) | 0.97 |
| (pg/mL) | | | | | |
| Vitamin D-Binding Protein (VDBP) (ug/mL) | 243 | (193, 288.5) | 249 | (184, 313) | 0.50 |
| Vitamin K-Dependent Protein S (VKDPS) (ug/mL) | 13 | (9.8, 17.5) | 14 | (11, 16.8) | 0.28 |
| Vitronectin (ug/mL) | 407 | (341.5, 506.5) | 462 | (351, 572.8) | 0.06 |
| von Willebrand Factor (vWF) (ug/mL) | 164 | (132, 202.5) | 131 | (95.3, 182) | 0.002 |

Six predictors were present in the final model (Table 6, Example 1, AKI 026e): four (history of diabetes type 2, blood urea nitrogen to creatinine ratio, C-reactive protein, and osteopontin) had a positive or direct association with AKI risk, e.g. biomarker levels were higher or presence of condition such as history of diabetes type 2 in association with AKI risk; while two (CD5 antigen-like and Factor VII) had a negative, or indirect, association with AKI risk, e.g. biomarker levels were lower in association with AKI risk. The final model had a cross-validated area under the receiver operating characteristic curve (AUC) of 0.79 for predicting procedural AKI, and an in-sample AUC of 0.82 (P<0.001). The optimal score cut-off had 77% sensitivity, 75% specificity, and a negative predictive value of 98% for procedural AKI. An elevated score was predictive of procedural AKI in all subjects (odds ratio=9.87; P<0.001). We describe a clinical and proteomics-supported biomarker model with high accuracy for predicting procedural AKI in patients undergoing coronary angiography (CASABLANCA; NCT00842868).

To date, machine learning for prediction of AKI in patients undergoing coronary angiography has not yet been studied. As such, we hypothesized that a proteomics-based and artificial intelligence-driven biomarker approach together with clinical risk factors would predict procedural AKI risk in patients enrolled in the Catheter Sampled Blood Archive in Cardiovascular Diseases (CASABLANCA) undergoing coronary angiographic procedures with or without interventions for various acute and non-acute indications.

### Methods

All study procedures were approved by the Partners Healthcare Institutional Review Board and carried out in accordance with the Declaration of Helsinki.

The design of the CASABLANCA (NCT NCT00842868) study has been detailed previously [48]. Briefly, 1251 patients undergoing coronary and/or peripheral angiography with or without intervention between 2008 and 2011 were prospectively enrolled at the Massachusetts General Hospital in Boston, Massachusetts. Patients were referred for angiography for various acute and non-acute indications. Of the 1251 patients enrolled, patients who did not undergo a coronary angiogram, patients who had a history of renal replacement therapy, those with missing blood urea nitrogen or creatinine values, and those with an insufficient quantity of sample were excluded. This left 889 patients undergoing coronary angiography with available blood samples.

After informed consent was obtained, detailed clinical and historical variables were recorded using a standardized case report form at the time of the angiographic procedure. This case report form included more than 100 clinical variables acquired at the time of study entry as well as results of coronary angiography. Angiographic results were based on visual interpretation by the operator, verified via the catheterization report.

Median follow-up was 4 years, with a maximum follow up of 6 years. Follow up was complete for all patients. Processes for identification and adjudication of clinical endpoints were as previously described [48] and included review of medical records as well as phone follow up with patients and/or managing physicians and was performed by physicians blinded to biomarker concentrations. The Social Security Death Index and/or postings of death announcements were used to confirm vital status. A detailed definition of endpoints for CASABLANCA was previously published [48].

Specific to this analysis, procedural AKI was defined as an abrupt reduction in kidney function with an absolute increase in serum creatinine of more than or equal to 0.3 mg/dL, a percentage increase in serum creatinine of >50%, or a reduction in urine output (documented oliguria of <0.5 mL/kg per hour for >6 hours), within 7 days after contrast exposure.

Baseline characteristics between those who developed procedural AKI and those who did not were compared. Dichotomous variables were compared using Fisher's exact test, while continuous variables were compared using t-test or Wilcox Rank Sum test.

A total of 15 mL of blood was obtained immediately before the angiographic procedure through a centrally-placed vascular access sheath. The blood was immediately centrifuged for 15 minutes, serum and plasma aliquoted on ice, and frozen at −80° C. until biomarker measurement. The samples for this study were analyzed after the first freeze-thaw cycle for baseline protein marker values only. Using Luminex xMAP technology, which is a bead-based multiplexed immunoassay system in a microplate format, 113 biomarkers in blood were measured (Table 3) from 889 patients undergoing coronary angiographic procedures for various indications.

A complete case analysis was performed; blood urea nitrogen or creatinine values were missing with some patients (n=167), so these patients were removed from the analysis. One other patient was removed from the analysis for having an insufficient quantity of sample, leaving 889 samples available for analysis. For any protein marker result that was below the limit of detection, we utilized a standard approach of imputing concentrations 50% below the limit of detection.

To facilitate the machine learning analysis, the concentrations for all proteins underwent the following transformations: (1) they were log-transformed to achieve a normal distribution; (2) outliers were clipped at the value of three times the median absolute deviation; and (3) the values were re-scaled to a distribution with zero mean and unit variance. The starting sets of variables consisted of all 113 proteins as well as clinical factors in the CASABLANCA dataset that were chosen for their possible clinical relevance. Clinical and biomarker predictors of AKI were identified using least-angle regression [49]. In this method, factors were included in the model one at a time, with their coefficients determined by their correlation with the outcome. This was repeated until all factors were included in the model, and the step at which the performance plateaued resulted in our initial panel of interest. Starting with this panel of interest, predictive analyses were run on the training set using least absolute shrinkage and selection operator [50](LASSO) with logistic regression, predicting the outcome of procedural AKI using only the variables in the panel of interest. This model-development process was done via Monte Carlo cross validation, using 400 iterations with an 80:20 (training:test) split. If the performance of the least contributing variable in the panel was not statistically significant, it was removed from the panel and the analysis repeated until the predictive contribution of all variables was statistically significant. With the final panel, its performance using the MCCV process described above was evaluated. Its in-sample performance using a final prognostic model developed on all of the available data with LASSO with logistic regression was determined. A cutoff was determined using the optimal Youden's index.

In all statistical analyses, a 2-tailed P value of <0.05 was considered statistically significant. All analyses were performed using the R statistical computing platform, Version 3.4.4.

### Results

Forty-three (4.8%) patients developed procedural AKI. Those who developed procedural AKI were older (70 vs. 67 years of age, p=0.04) and more likely to have prevalent diabetes mellitus (41.9% vs. 23.5%, p=0.01) or CKD (20.9% vs. 10.4%, p=0.04) (Table 4). Those who developed procedural AKI also had lower left ventricular ejection fraction at baseline (50.0% vs. 56.6%, p=0.04) and a higher percentage of them were prescribed an angiotensin converting enzyme inhibitor (ACEi)/angiotensin receptor blocker (ARB) compared to those who did not develop AKI (72.1% vs. 53.6%, respectively, p=0.02) (Table 4).

As expected, those who developed procedural AKI had higher blood urea nitrogen (BUN) (21 vs. 18 mg/dL, p=0.006) and BUN/creatinine ratio (20.1 vs 17.8, p=0.04) and lower eGFR (77.7 vs 99.2 mL/min/1.73 m², p<0.001) and hemoglobin (12.3 vs. 13.3 g/dL, p<0.001) at baseline compared to those who did not develop procedural AKI. They also had higher baseline concentrations of C-reactive protein (CRP) (8.8 vs. 3.5 mg/L) and osteopontin (43 vs. 27 ng/mL) and lower concentrations of Factor VII (350 vs. 468 ng/mL) and CD5 antigen-like (3600 vs. 3755 pg/mL) compared to those who did not develop procedural AKI (Table 4).

Following the machine learning-driven approach to panel development used herein, six predictors were present in the final model (Table 6, AKI 26e: four (history of diabetes, BUN/creatinine ratio, CRP, and osteopontin) had a positive or direct association with AKI risk; while two (CD5 antigen-like and Factor VII) had a negative or indirect association with AKI risk. Using the model-building procedure described above for subsets of variables, the addition of each biomarker provided a statistically significant improvement in the AUC and the likelihood ratio, while decreasing the AIC and the BIC (Table 5).

The final model had a cross-validated area under the receiver operating characteristic curve (AUC) of 0.79 and an in-sample AUC of 0.82 (p<0.001) for predicting procedural AKI. The optimal score cut-off had 77% sensitivity, 75% specificity, and a negative predictive value of 98% for procedural AKI (FIG. 1). An elevated score was predictive of procedural AKI in all subjects (odds ratio=9.87; p<0.001).

### Discussion

Amongst a typical population of 889 patients undergoing coronary angiography with or without interventions for various acute and non-acute indications, 4.8% of patients developed procedural AKI. A model was created that included 6 predictors of AKI: four (history of diabetes, BUN to creatinine ratio, CRP, and osteopontin) had a positive or direct association with AKI risk; while two (CD5 antigen-like and Factor VII) had a negative or indirect association with AKI risk. The final model had a high accuracy for predicting procedural AKI in patients undergoing coronary angiography.

The rationale for this study is based on the fact that AKI following coronary angiographic procedures is associated with significant morbidity and mortality that has potential to alter patient management if predicted early [51,52]. Ability to predict onset of AKI earlier might alter management in efforts toward its prevention, such as alteration of angiography plans (i.e., minimizing dye exposure and employing bi-plane angiography, for example), avoidance of nephrotoxins, or pre-procedure hydration. In those at risk for CKD progression due to presence of comorbidities such as diabetes and HF, interventions might be considered to reduce its incidence including hydration, better control of such comorbidities, avoidance of nephrotoxins, and consideration of delaying elective angiography plans until such comorbidities are better managed.

Prior work has examined this question, mostly based on clinical variables. Among patients in the Minnesota Registry of Interventional Cardiac Procedures, diabetes, increased age, higher dose and route of contrast administration, HF, hypertension, periprocedural shock, baseline anemia, post-procedural drop in hematocrit, use of nephrotoxins, volume depletion, increased creatinine kinase-muscle/brain enzyme, and need for cardiac surgery after contrast exposure were associated with increased risk of procedural AKI [53]. Mehran and colleagues developed a simple risk score that included pre- and peri-procedural risk factors including hypotension, intra-aortic balloon pump, HF, CKD, diabetes, age >75 years, anemia, and volume of contrast with good discriminative power (c-statistic 0.67) [4]. In another AKI risk prediction model developed by Brown and colleagues, pre-procedural serum creatinine, HF, and diabetes accounted for >75% of the predictive model [53, 54].

While BUN and serum creatinine are most often used to predict procedural AKI, they are not very sensitive or specific for the diagnosis of AKI because they are affected by many renal and non-renal factors that are independent of kidney injury or kidney function [55]. As such, several protein markers and protein marker panels with and without clinical risk factors have been examined to more accurately predict AKI. The risk prediction model herein included the BUN/creatinine ratio in addition to clinical and biomarker risk factors to better predict procedural AKI.

Inflammation may play an important role in presence and severity of AKI. C reactive protein (CRP) is an acute-phase protein of hepatic origin that is a marker of inflammation synthesized in response to factors released by macrophages and adipocytes [56]. CRP has been associated with cardiovascular risk [57] and has also been associated with renal dysfunction [58]. Tang and colleagues demonstrated that elevated serum CRP concentrations were associated with increased serum creatinine and urea concentrations (p<0.01) in patients with AKI; CRP concentrations subsequently fell after recovery from AKI [59]. In older patients with AKI, CRP was an independent risk factor for mortality [60]. CRP has also been studied for its ability to predict risk for AKI. In a study of 1,656 patients undergoing coronary artery bypass grafting, pre-operative CRP concentrations predicted post-operative AKI and mortality; the addition of CRP to an existing risk model improved net reclassification and discrimination [61]. That finding herein that concentrations of CRP as a predictor of procedural AKI is consistent with this body of evidence.

Osteopontin is an extracellular matrix protein and proinflammatory cytokine thought to facilitate the recruitment of monocytes/macrophages and to mediate cytokine secretion in leukocytes. It plays a role in many physiological and pathological processes, including biomineralization, tissue remodeling, and inflammation [62]. It is found mainly in the loop of Henle and distal nephrons in normal kidneys and can be upregulated in all tubular and glomerular segments following kidney damage, and may also have a role in renal repair [63]. In the last several years, the role of osteopontin in the pathogenesis of diabetic nephropathy has been explored [62]. Osteopontin has been reported to be highly expressed in the tubular epithelium of the renal cortex and in glomeruli in rat and mouse models of diabetic nephropathy [64] and in humans, plasma osteopontin concentrations are independently associated with the presence and severity of diabetic nephropathy [65]. In a study of critically ill patients with AKI requiring renal replacement therapy, concentrations of osteopontin were significantly higher than in critically ill patients without AKI. Additionally, osteopontin concentrations were found to be a strong predictor of mortality with an AUC of 0.82 (95% confidence interval [CI]: 0.74-0.89; p<0.0001), sensitivity of 100%, and specificity of 61% for a cutoff value of 577 ng/ml [66].

CD5 antigen-like is a secreted protein encoded by the CD5L gene that acts as a key regulator of lipid synthesis. It is mainly expressed by macrophages in lymphoid and inflamed tissues and regulates mechanisms in inflammatory responses, such as infection or atherosclerosis [67]. Recently, in patients with diabetes, CD5 antigen-like has been identified as a protein marker that may be able to improve rapid decline in kidney function independently of recognized clinical risk factors (odds ratio 0.52, 95% CI 0.29-0.93) and improved model performance in predicting other indices of rapid eGFR decline [68].

Data regarding Factor VII and its ability to predict kidney dysfunction are scarce; however, it is well-established as a marker of hypercoagulability and persistence of inflammatory response [69]. Sublethal injury to kidney cells may affect renal blood flow and be associated with the complications of impaired coagulability and intra-organ hemorrhage. Low levels of Factor VII could exacerbate impaired coagulability and intra-organ hemorrhage. Further, in a subset of patients that were admitted to the hospital and developed AKI, Factor VII was decreased compared to healthy controls [70].

The AKI risk prediction model described herein incorporated clinical and biomarker predictors all known to affect renal function and was based on an unbiased, machine learning approach for selection of model variables. Major advantages of the cohort herein are its detailed characterization and experience working within this database, although limitations to the study exist. The CASABLANCA cohort was predominantly male, Caucasian, and representative of patients in a tertiary care referral center. Additionally, not included was the volume of contrast dye used during the coronary angiographic procedures, which clearly affects risk for AKI development. In contrast to measures of kidney function (such as creatinine or eGFR), a theoretical advantage of the risk prediction model herein is the potential detection of AKI prior to change in measures of kidney function and the inclusion of several predictors associated with AKI development. Earlier prediction of AKI can allow for adjustments in patient/care management that might help to mitigate risk for severe kidney dysfunction [71].

In conclusion, in a typical at-risk population undergoing coronary angiography for various acute and non-acute indications, described herein is a clinical and proteomics-supported biomarker model with high accuracy for predicting procedural AKI in patients undergoing coronary angiography. The ability to predict AKI may allow for earlier interventions in at-risk patients to reduce future AKI risk.

TABLE 4
Baseline characteristics of those who developed acute kidney injury
compared to those who did not.

| | With Procedural | | Without | | |
|---|---|---|---|---|---|
| Variable | AKI | | Procedural AKI | | p |
| Age (years) | 70 | | 67 | | 0.04 |
| Male sex | 31 (72.1%) | | 607 (71.7%) | | 1 |
| Caucasian race | 42 (97.7%) | | 785 (92.8%) | | 0.36 |
| Body mass index (kg/m²) | 28.7 | | 29.1 | | 0.67 |
| Heart rate (beat/min) | 70 | | 69 | | 0.67 |
| Systolic blood pressure (mmHg) | 137 | | 136 | | 0.87 |
| Diastolic blood pressure (mmHg) | 72 | | 72 | | 0.66 |
| Smoker | 4 (9.5%) | | 120 (14.3%) | | 0.50 |
| Atrial fibrillation/flutter | 8 (18.6%) | | 171 (20.2%) | | 1 |
| Hypertension | 37 (86.0%) | | 608 (71.9%) | | 0.05 |
| Coronary artery disease | 26 (60.5%) | | 431 (50.9%) | | 0.27 |
| Prior myocardial infarction | 13 (30.2%) | | 205 (24.2%) | | 0.37 |
| Heart failure | 12 (27.9%) | | 174 (20.6%) | | 0.25 |
| Peripheral artery disease | 13 (30.2%) | | 153 (18.1%) | | 0.07 |
| Chronic obstructive pulmonary disease | 11 (25.6%) | | 145 (17.2%) | | 0.15 |
| Diabetes type I/type II | 18 (41.9%) | | 199 (23.5%) | | 0.01 |
| CVA/TIA | 7 (16.3%) | | 85 (10.0%) | | 0.20 |
| Chronic kidney disease | 9 (20.9%) | | 88 (10.4%) | | 0.04 |
| Prior angioplasty | 6 (14.0%) | | 85 (10.0%) | | 0.43 |
| Prior stent | 17 (39.5%) | | 232 (27.4%) | | 0.12 |
| Prior coronary artery bypass grafting | 9 (20.9%) | | 163 (19.3%) | | 0.84 |
| Prior percutaneous coronary | 16 (37.2%) | | 253 (29.9%) | | 0.31 |
| intervention | | | | | |
| ACEi/ARB | 31 (72.1%) | | 451 (53.6%) | | 0.02 |
| Beta blockers | 27 (62.8%) | | 589 (69.8%) | | 0.40 |
| Aldosterone antagonists | 2 (4.7%) | | 30 (3.6%) | | 0.67 |
| Loop diuretics | 15 (34.9%) | | 180 (21.3%) | | 0.06 |
| Nitrates | 14 (32.6%) | | 166 (19.7%) | | 0.05 |
| Calcium channel blockers | 13 (30.2%) | | 193 (22.9%) | | 0.27 |
| Statins | 29 (67.4%) | | 612 (72.6%) | | 0.49 |
| Aspirin | 31 (72.1%) | | 643 (76.4%) | | 0.58 |
| Warfarin | 9 (20.9%) | | 127 (15.0%) | | 0.28 |
| Clopidogrel | 12 (27.9%) | | 188 (22.3%) | | 0.45 |
| Left ventricular ejection fraction (%) | 50.0 | | 56.6 | | 0.04 |
| Sodium (mEq/L) | 138.7 | | 139.3 | | 0.27 |
| Blood urea nitrogen (mg/dL) | 21 | (16.5, 30) | 18 | (14, 23) | 0.006 |
| Blood urea nitrogen/creatinine | 20.1 | | 17.8 | | p = 0.04 |
| Creatinine (mg/dL) | 1.2 | (0.9, 1.5) | 1.1 | (0.9, 1.3) | 0.29 |
| eGFR (CKD-EPI) (mL/min/1.73 m²) | 77.7 | (63.8, 95.0) | 99.2 | (75.6, 110.7) | <0.001 |
| Hemoglobin A1c | 6.4 | (6.2, 7.4) | 6.1 | (5.6, 6.9) | 0.27 |
| Hemoglobin (g/dL) | 12.3 | (1.5) | 13.3 | (1.7) | <0.001 |
| C-reactive protein (mg/L) | 8.8 | (3.8, 22.5) | 3.5 | (1.5, 9.1) | <0.001 |
| CD5 antigen-like (ng/mL) | 3600 | (2695, 5370) | 3755 | (2860, 5097.5) | 0.77 |
| Factor VII (ng/mL) | 350 | (290.5, 523) | 468 | (360, 588.75) | 0.005 |
| Osteopontin (ng/mL) | 43 | (31.5, 66) | 27 | (20, 41) | <0.001 |

AKI = acute kidney injury,
CVA/TIA = cerebrovascular accident/transient ischemic attack,
ACEi/ARB = angiotensin converting enzyme inhibitor/angiotensin receptor blocker,
eGFR = estimated glomerular filtration rate,
CKD-EPI = chronic kidney disease-epidemiology.

TABLE 5
Procedural acute kidney injury risk score model
calibration and goodness of fit.

| Panel | AIC | BIC | H-L p |
|---|---|---|---|
| Diabetes | 340.6 | 350.2 | 1 |
| Diabetes + BUN/Cr | 338.0 | 352.4 | 0.30 |
| Diabetes + BUN/Cr + osteopontin | 319.4 | 338.6 | 0.77 |
| Diabetes + BUN/Cr + osteopontin + CRP | 313.5 | 337.4 | 0.71 |
| Diabetes + BUN/Cr + osteopontin + CRP + | 309.1 | 337.8 | 0.77 |
| Factor VII | | | |
| Diabetes + BUN/Cr + osteopontin + CRP + | 305.0 | 338.5 | 0.96 |
| Factor VII + CD5 antigen-like | | | |

### Example 2: Further Demonstration of Methods Employing Clinical and Protein Marker Analysis for the Procedural Acute Kidney Injury

Table 6 is a chart of the different panels comprising protein markers and optionally clinical variables with corresponding AUCs for the given outcome. These reflect aforementioned Example 1, as well as additional panels in Examples 3, 4, and 5 generated using the methods and analysis provided herein.

### Example 3: Further Demonstration of Methods Employing Clinical and Protein Marker Analysis for the Procedural Acute Kidney Injury

Following the machine learning-driven approach to panel development used herein, seven predictors were present in the final model (Table 6, AKI 27e: five (history of diabetes, BUN/creatinine ratio, CRP, Kidney Injury Molecule 1, and osteopontin) had a positive or direct association with AKI risk; while two (CD5 antigen-like and Factor VII) had a negative or indirect association with AKI risk.

The final model had a cross-validated area under the receiver operating characteristic curve (AUC) of 0.78 and an in-sample AUC of 0.82 (p<0.001) for predicting procedural AKI. The optimal score cut-off had 74% sensitivity, 76% specificity, and a negative predictive value of 98% for procedural AKI (FIG. 2).

### Example 4: Further Demonstration of Methods Employing Clinical and Protein Marker Analysis for the Procedural Acute Kidney Injury

Following the machine learning-driven approach to panel development used herein, five predictors were present in the final model (Table 6, AKI 032e: all five (history of diabetes, BUN/creatinine ratio, CRP, Kidney Injury Molecule 1, and osteopontin) had a positive or direct association with AKI risk.

The final model had a cross-validated area under the receiver operating characteristic curve (AUC) of 0.74 and an in-sample AUC of 0.77 (p<0.001) for predicting procedural AKI. The optimal score cut-off had 63% sensitivity, 81% specificity, and a negative predictive value of 98% for procedural AKI (FIG. 3).

### Example 5: Further Demonstration of Methods Employing Clinical and Protein Marker Analysis for the Procedural Acute Kidney Injury

Following the machine learning-driven approach to panel development used herein, three predictors were present in the final model (Table 6, AKI 52e: all three (BUN/creatinine ratio, and N-terminal prohormone of brain natriuretic peptide) had a positive or direct association with AKI risk.

The final model had a cross-validated area under the receiver operating characteristic curve (AUC) of 0.75 and an in-sample AUC of 0.76 (p<0.001) for predicting procedural AKI. The optimal score cut-off had 81% sensitivity, 67% specificity, and a negative predictive value of 99% for procedural AKI (FIG. 4).

TABLE 6
Performance of Different Panels Comprising Protein markers and
Optionally Clinical Variables with Corresponding AUCs and Figures

| | | | Cross | In | |
|---|---|---|---|---|---|
| | Test | | Validated | Sample/Entire | |
| | Outcome/ | Protein markers | Mean AUCs | Population | |
| | Positive | & Clinical | (rounded to | (rounded to | Figure |
| Analysis # | Endpoint | Variables | nearest 0.00) | nearest 0.00) | Reference |
| Prognostic and/or Monitoring | | | | | |
| Therapeutic Effect and/or | | | | | |
| Identification for Clinical | | | | | |
| Trial | | | | | |
| AKI 026e | Prognosis for | CD5 Antigen Like, | 0.79 | 0.82 | 1 |
| Example 1 | Acute Kidney | C Reactive Protein, | | (rounded from | |
| | Injury | Factor VII, | | 0.816) | |
| | | Osteopontin, | | | |
| | | BUN:Creatinine | | | |
| | | Ratio, History of | | | |
| | | Diabetes type 2 | | | |
| AKI 027e | Prognosis for | CD5 Antigen Like, | 0.78 | 0.82 | 2 |
| Example 3 | Acute Kidney | C Reactive Protein, | | (rounded from | |
| | Injury | Factor VII, Kidney | | 0.816) | |
| | | Injury Molecule 1, | | | |
| | | Osteopontin, | | | |
| | | BUN:Creatinine | | | |
| | | Ratio, History of | | | |
| | | Diabetes type 2 | | | |
| AKI 032e | Prognosis for | C Reactive Protein, | 0.74 | 0.77 | 3 |
| Example 4 | Acute Kidney | Kidney Injury | | (rounded from | |
| | Injury | Molecule 1, | | 0.765) | |
| | | Osteopontin, | | | |
| | | BUN:Creatinine | | | |
| | | Ratio, History of | | | |
| | | Diabetes type 2 | | | |
| AKI 052e | Prognosis for | C Reactive Protein, | 0.75 | 0.76 | 4 |
| Example 5 | Acute Kidney | N-terminal | | (rounded from | |
| | Injury | prohormone of | | 0.761) | |
| | | brain natriuretic | | | |
| | | peptide, | | | |
| | | BUN:Creatinine | | | |
| | | Ratio | | | |

### Example 3: Mathematical Determinations

A diagnostic or prognostic algorithm in the form of a linear model is represented by a mathematical formula in the following form:

$$Diagnostic score = a + {b}_{1} {x}_{1} + {b}_{2} {x}_{2} + … + {b}_{n} {x}_{n}$$

where x₁ through xₙ are the model inputs (such as protein concentrations or clinical information), b₁ through bₙ are the coefficients of the model, and a is the “intercept” term.

Here is an example of a diagnostic algorithm in the form of a linear model, involving three protein concentrations as inputs:

$$Diagnostic score = 3.5 + 1.8 {x}_{1} + 2.9 {x}_{2} - 1.3 {x}_{3}$$

In this case, proteins 1 and 2 have a positive effect on disease risk (higher concentrations result in higher risk), as the coefficients are positive (as indicated by the plus sign in the model preceding the coefficients). Protein 3 has an inverse effect on disease risk (lower concentrations results in higher risk), as the coefficient is negative (as indicated by the minus sign preceding the coefficient).

If a patient has concentrations of 0.5 (protein 1), 2.5 (protein 2) and 1.5 (protein 3), then we enter those concentrations into the model and get the following:

$$Diagnostic score = 3.5 + ( 1.8 * 0.5 ) + ( 2.9 * 2.5 ) - ( 1.3 * 1.5 ) = 9.7$$

The model would have cut-offs that would enable one to place 9.7 as either positive, intermediate, or negative result and allow for a determination of a diagnosis (or prognosis) of an outcome or event.

### REFERENCES

1. Damluji A, Cohen M G, Smairat R, Steckbeck R, Moscucci M, Gilchrist I C. The incidence of acute kidney injury after cardiac catheterization or PCI: A comparison of radial vs. femoral approach. International Journal of Cardiology 2014; 173:595-597.
2. Azzalini L, Candilio L, McCullough P A, Colombo A. Current Risk of Contrast-Induced Acute Kidney Injury After Coronary Angiography and Intervention: A Reappraisal of the Literature. Canadian Journal of Cardiology 2017; 33:1225-1228.
3. Prigent A. Monitoring Renal Function and Limitations of Renal Function Tests. Seminars in Nuclear Medicine 2008; 38:32-46.
4. Mehran R, Aymong E D, Nikolsky E, Lasic Z, Iakovou I, Fahy M, Mintz G S, Lansky A J, Moses J W, Stone G W, Leon M B, Dangas G. A simple risk score for prediction of contrast-induced nephropathy after percutaneous coronary intervention: Development and initial validation. Journal of the American College of Cardiology 2004; 44:1393-1399.
5. Jarai R, Dangas G, Huber K, Xu K, Brodie B R, Witzenbichler B, Metzger D C, Radke P W, Yu J, Claessen B E, Genereux P, Mehran R, Stone G W. B-type Natriuretic Peptide and Risk of Contrast-Induced Acute Kidney Injury in Acute S T-Segment-Elevation Myocardial Infarction. A Substudy from the HORIZONS—AMI Trial 2012; 5:813-820.
6. Gurm H S, Seth M, Kooiman J, Share D. A Novel Tool for Reliable and Accurate Prediction of Renal Complications in Patients Undergoing Percutaneous Coronary Intervention. Journal of the American College of Cardiology 2013; 61:2242-2248.
7. Koyner J L, Carey K A, Edelson D P, Churpek M M. The Development of a Machine Learning Inpatient Acute Kidney Injury Prediction Model*. Critical Care Medicine 2018; 46:1070-1077.
8. Mohamadlou H, Lynn-Palevsky A, Barton C, Chettipally U, Shieh L, Calvert J, Saber N R, Das R. Prediction of Acute Kidney Injury With a Machine Learning Algorithm Using Electronic Health Record Data. Canadian Journal of Kidney Health and Disease 2018; 5:2054358118776326.
9. Devarajan P. Genomic and Proteomic Characterization of Acute Kidney Injury. Nephron 2015; 131:85-91.
10. Bennett M R, Devarajan P. Proteomic analysis of acute kidney injury: biomarkers to mechanisms. Proteomics Clinical applications 2011; 5:67-77.
11. Konvalinka A. Urine proteomics for acute kidney injury prognosis: another player and the long road ahead. Kidney International 2014; 85:735-738.
12. Sambrook, et al., Molecular Cloning: A Laboratory Manual (3rd Edition, 2001).
13. Sambrook, et al., Molecular Cloning: A Laboratory Manual (2nd Edition, 1989).
14. Maniatis et al., Molecular Cloning: A Laboratory Manual (1982);
15. Ausubel et al. Current Protocols in Molecular Biology (John Wiley and Sons, updated July 2008)
16. Short Protocols in Molecular Biology: A Compendium of Methods from Current Protocols in Molecular Biology, Greene Pub. Associates and Wiley-Interscience
17. Glover, DNA Cloning: A Practical Approach, vol. I & II (IRL Press, Oxford, 1985)
18. Anand, Techniques for the Analysis of Complex Genomes, (Academic Press, New York, 1992)
19. Transcription and Translation (B. Hames & S. Higgins, Eds., 1984)
20. Perbal, A Practical Guide to Molecular Cloning (1984)
21. Harlow and Lane, Antibodies, (Cold Spring Harbor Laboratory Press, Cold Spring Harbor, N. Y., 1998)
22. Kohler et al., Nature, 256: 495 (1975)
23. Clackson et al., Nature, 352: 624-628 (1991)
24. Marks et al., J. Mol. Biol., 222: 581-597 (1991)
25. U.S. Pat. No. 4,816,567
26. Morrison et al., Proc. Natl. Acad. Sci. USA, 81: 6851-6855 (1984)
27. Harlow and Lane, eds. (Antibodies: A Laboratory Manual (1988) Cold Spring Harbor Laboratory Press, Cold Spring Harbor, N.Y.)
28. McVey, J. H. Tissue factor pathway. Baillieres Clin Haematol 7, 469-484 (1994).
29. Xie, Q. et al. The ratio of CRP to prealbumin levels predict mortality in patients with hospital-acquired acute kidney injury. BMC Nephrol 12, 30, doi:10.1186/1471-2369-12-30 (2011).
30. Diao, H. et al. Osteopontin as a mediator of NKT cell function in T cell-mediated liver diseases. Immunity 21, 539-550, doi:10.1016/j.immuni.2004.08.012 (2004).
31. Ling-Na Zheng et al., 2011, J. of Analytical Atomic Spectrometry, 26, 1233-1236
32. Vaudel, M., et al., 2010, Proteomics, Vol. 10: 4
33. Pan, S., 2009 J. Proteome Research, February; 8(2):787-97
34. Westermeier and Marouga, 2005, Bioscience Reports, Vol. 25, Nos. ½
35. Carr and Anderson, 2008, Clinical Chemistry. 54:1749-1752
36. Aebersold and Mann, 2003, Nature, Vol. 422
37. US2008/0202927
38. US2014/0256573
39. US2016/0153980
40. WO2016/001795
41. US2008/0185295
42. US2010/0047901
43. US2010/0231242
44. US2011/0154648
45. US2013/0306491
46. US2010/0329929
47. US2013/0261009
48. Han, S. S. et al. C-Reactive Protein Predicts Acute Kidney Injury and Death After Coronary Artery Bypass Grafting. Ann Thorac Surg 104, 804-810, doi:10.1016/j.athoracsur.2017.01.075 (2017).
49. Efron B, Hastie T, Johnstone I, Tibshirani R. Least angle regression. 2004:407-499.
50. Tibshirani R. Regression shrinkage and selection via the lasso: a retrospective. J R Stat Soc Series B Stat Methodol. 2011; 73:273-282.
51. Anavekar N S, McMurray J J V, Velazquez E J, Solomon S D, Kober L, Rouleau J-L, White H D, Nordlander R, Maggioni A, Dickstein K, Zelenkofske S, Leimberger J D, Califf R M, Pfeffer M A. Relation between Renal Dysfunction and Cardiovascular Outcomes after Myocardial Infarction. New England Journal of Medicine 2004; 351:1285-1295.
52. James M T, Ghali W A, Knudtson M L, Ravani P, Tonelli M, Faris P, Pannu N, Manns B J, Klarenbach S W, Hemmelgarn B R. Associations Between Acute Kidney Injury and Cardiovascular and Renal Outcomes After Coronary Angiography. Circulation 2011; 123:409-416.
53. Kagan A, Sheikh-Hamad D. Contrast-induced Kidney Injury: Focus on Modifiable Risk Factors and Prophylactic Strategies. Clinical Cardiology 2010; 33:62-66.
54. Brown J R, DeVries J T, Piper W D, Robb J F, Hearne M J, Ver Lee P M, Kellet M A, Watkins M W, Ryan T J, Silver M T, Ross C S, MacKenzie T A, O'Connor G T, Malenka D J. Serious renal dysfunction after percutaneous coronary interventions can be predicted. American Heart Journal 2008; 155:260-266.
55. Edelstein C L. Biomarkers of Acute Kidney Injury. Advances in Chronic Kidney Disease 2008; 15:222-234.
56. Lau D C W, Dhillon B, Yan H, Szmitko P E, Verma S. Adipokines: molecular links between obesity and atheroslcerosis. American Journal of Physiology-Heart and Circulatory Physiology 2005; 288:H2031-H2041.
57. Shrivastava A K, Singh H V, Raizada A, Singh S K. C-reactive protein, inflammation and coronary heart disease. The Egyptian Heart Journal 2015; 67:89-97.
58. Pecoits-Filho R, Heimburger O, Bárány P, Suliman M, Fehrman-Ekholm I, Lindholm B, Stenvinkel P. Associations between circulating inflammatory markers and residual renal function in CRF patients. American Journal of Kidney Diseases 2003; 41:1212-1218.
59. Tang Y, Huang Xiao R, Lv J, Chung Arthur C-K, Zhang Y, Chen J-Z, Szalai Alexander J, Xu A, Lan Hui Y. C-reactive protein promotes acute kidney injury by impairing G<sub>1</sub>/S-dependent tubular epithelium cell regeneration. Clinical Science 2014; 126:645-659.
60. Kayatas K, Sahin G, Tepe M, Kaya Z E, Apaydin S, Demirtung R. Acute kidney injury in the elderly hospitalized patients. Renal Failure 2014; 36:1273-1277.
61. Han S S, Kim D K, Kim S, Chin H J, Chae D-W, Na K Y. C-Reactive Protein Predicts Acute Kidney Injury and Death After Coronary Artery Bypass Grafting. The Annals of Thoracic Surgery 2017; 104:804-810.
62. Kahles F, Findeisen H M, Bruemmer D. Osteopontin: A novel regulator at the cross roads of inflammation, obesity and diabetes. Molecular Metabolism 2014; 3:384-393.
63. Taub P R, Borden K C, Fard A, Maisel A. Role of biomarkers in the diagnosis and prognosis of acute kidney injury in patients with cardiorenal syndrome. Expert review of cardiovascular therapy 2012; 10:657-667.
64. Fischer J W, Tschöpe C, Reinecke A, Giachelli C M, Unger T. Upregulation of osteopontin expression in renal cortex of streptozotocin-induced diabetic rats is mediated by bradykinin. Diabetes 1998; 47:1512-1518.
65. Yan X, Sano M, Lu L, Wang W, Zhang Q, Zhang R, Wang L, Chen Q, Fukuda K, Shen W. Plasma concentrations of osteopontin, but not thrombin-cleaved osteopontin, are associated with the presence and severity of nephropathy and coronary artery disease in patients with type 2 diabetes mellitus. Cardiovascular Diabetology 2010; 9:70-70.
66. Lorenzen J M, Hafer C, Faulhaber-Walter R, Kumpers P, Kielstein J T, Haller H, Fliser D. Osteopontin predicts survival in critically ill patients with acute kidney injury. Nephrology Dialysis Transplantation 2011; 26:531-537.
67. UniProt https://www.uniprot.org/uniprot/043866 Accessed 8/2/18.
68. Peters K E, Davis W A, I to J, Winfield K, Stoll T, Bringans S D, Lipscombe R J, Davis T M E. Identification of Novel Circulating Biomarkers Predicting Rapid Decline in Renal Function in Type 2 Diabetes: The Fremantle Diabetes Study Phase II. Diabetes Care 2017; 40:1548-1555.
69. Adams M J, Irish A B, Watts G F, Oostryck R, Dogra G K. Hypercoagulability in chronic kidney disease is associated with coagulation activation but not endothelial function. Thrombosis Research 2008; 123:374-380.
70. Agarwal B. et al. Hemostasis in patients with acute kidney injury secondary to acute liver failure. Kidney Int 84, 158-163, doi:10.1038/ki.2013.92 (2013)
71. Nijssen E C, Rennenberg R J, Nelemans P J, Essers B A, Janssen M M, Vermeeren M A, Ommen V v, Wildberger J E. Prophylactic hydration to protect renal function from intravascular iodinated contrast material in patients at high risk of contrast-induced nephropathy (AMACING): a prospective, randomised, phase 3, controlled, open-label, non-inferiority trial. The Lancet 2017; 389:1312-1322.

### P-Embodiments

Embodiment P-1. A method of determining risk of acute kidney injury in a subject, comprising:
(i) providing a biological sample from a subject suspected of having a risk of acute kidney injury,
(ii) applying the biological sample to an analytical device, to
   (a) detect the concentration of at least two protein markers in the sample;
   (b) normalize said concentration of protein markers against a synthetic quantification standard and
   (c) transform the normalized protein marker concentrations;
wherein the at least two protein markers are selected from those set forth in Table 1;
(iii) optionally, determining the status of at least one clinical variable or measurement for the subject, wherein the clinical variable or measurement is selected from those set forth in Table 2;
(iv) calculating a score using an algorithm based on the normalized, transformed protein markers determined in step (ii) and, optionally, the status of the clinical variable or marker determined in step (iii);
(v) classifying the score as a positive, intermediate, or negative result; and
(vi) determining a prognosis of acute kidney injury risk in a subject as indicated by the score.

Embodiment P-2. The method of Embodiment P-1, further comprising treating the subject based on the positive, intermediate, or negative score, wherein the treatment comprises a therapeutic intervention regimen.

Embodiment P-3. The method of Embodiment P-1, wherein the sample comprises plasma.

Embodiment P-4. The method of Embodiment P-1, wherein the at least two protein markers are selected from CD5 antigen like, C reactive protein, Factor VII, kidney injury molecule 1, and osteopontin; and wherein the optional step (iii) comprises determining blood urea nitrogen:creatinine ratio and the status of history of diabetes type 2.

Embodiment P-5. The method of any one of Embodiments P-1 to P-4 wherein the at least two protein markers are C-reactive protein, CD5 antigen-like, Factor VII, and osteopontin, and wherein the optional step (iii) comprises determining blood urea nitrogen:creatinine ratio and the status of history of diabetes type 2.

Embodiment P-6. The method of any of the preceding Embodiments, wherein the prognosis of acute kidney injury risk in the subject comprises a prognosis of abrupt reduction in kidney function.

Embodiment P-7. The method of any of Embodiments P-1 to P-6, wherein a positive score in the subject facilitates a determination by a medical practitioner of the need for one or more interventions selected from ultrasound, administration of pharmacological agents, hydration, delaying a cardiac catheterization or other dye-based procedure and avoidance of any drug or procedure with a known kidney risk.

Embodiment P-8. The method of any of Embodiments P-1 to P-6, wherein a negative score in the subject facilitates a determination by a medical practitioner of the need for one or more interventions selected from ongoing monitoring and management of peripheral and coronary risk factors, and proceeding with a cardiac catheterization or other dye-based procedure.

Embodiment P-9. The method of any of Embodiments P-1 to P-6, wherein an intermediate score in the subject facilitates a determination by a medical practitioner of the need for one or more interventions selected from further testing, proceeding with a cardiac catheterization or other dye-based procedure whereby dye usage is strictly limited, more frequent monitoring for risk factors and lifestyle modifications. Embodiment P-10. A method of administering a therapeutic intervention to a subject having acute kidney injury risk comprising:
(i) determining the subject's protein marker profile for a panel of protein markers comprising at least two protein markers selected from those set forth in Table 1;
(ii) optionally, determining the status of at least one clinical variable or measurement for the subject, wherein the clinical variable or measurement is selected from those set forth in Table 2;
(iii) assigning a score to the subject based on the protein marker profile in (i) and optionally the clinical variable of measurement in (ii) wherein the score is classified as positive, intermediate, and negative, said score algorithmically-derived from the normalized and mathematically transformed concentrations of protein markers in the subject's sample and optionally, the status of at least one clinical variable or measurement; and
(iv) administering to the subject a therapeutic intervention based on the positive, intermediate or negative score.

Embodiment P-11. A method of detecting two or more protein markers in a subject having diabetes type 2 and/or that is suspected of having acute kidney injury risk, the method comprising:
(i) selecting a subject that has diabetes type 2 and/or that is suspected of having acute kidney injury risk;
(ii) providing a biological sample from the subject;
(iii) applying the biological sample to an analytical device, and
(iv) detecting the concentration of at least two protein markers from Table 1.

Embodiment P-12. The method of Embodiment P-11, further comprising:
(v) calculating a prognostic score based on the concentration of protein markers determined in step (iv);
(vi) classifying the prognostic score as a positive, intermediate, or negative result; and (vii) determining acute kidney injury risk in a subject as indicated by the prognostic score.

Embodiment P-13. The method of any one of Embodiments P-11 or P-12, wherein the at least two protein markers are selected from CD5 antigen like, C reactive protein, Factor VII, kidney injury molecule 1, and osteopontin and further wherein blood urea nitrogen:creatinine ratio is determined.

Embodiment P-14. The method of any one of claims Embodiments P-11 to P-12, wherein the at least two protein markers are CD5 antigen-like, C-reactive protein, Factor VII, and osteopontin and further wherein blood urea nitrogen:creatinine ratio is determined.

Embodiments P-15. The method of any one of Embodiments P-11 to P-12, wherein the at least two protein markers are CD5 antigen-like, C-reactive protein, Factor VII, kidney injury molecule 1, osteopontin and further wherein blood urea nitrogen:creatinine ratio is determined.

Embodiment P-16. The method of any one of Embodiments P-11 to P-12, wherein the at least two protein markers are C-reactive protein, kidney injury molecule 1, and osteopontin and further wherein blood urea nitrogen:creatinine ratio is determined.

Embodiment P-17. The method of any of Embodiments P-11 to P-16, wherein the determination of acute kidney injury risk in the subject comprises a prognosis of abrupt reduction in kidney function.

Embodiment P-18. The method of any of Embodiments P-11 to P-16, wherein a positive prognostic score in the subject facilitates a determination by a medical practitioner of the need for one or more interventions selected from ultrasound, administration of pharmacological agents, hydration, delaying a cardiac catheterization or other dye-based procedure and avoidance of any drug or procedure with a known kidney risk.

Embodiment P-19. The method of any of Embodiments P-11 to P-16, wherein a negative prognostic score in the subject facilitates a determination by a medical practitioner of the need for one or more interventions selected from ongoing monitoring and management of peripheral and coronary risk factors, and proceeding with a cardiac catheterization or other dye-based procedure.

Embodiment P-20. The method of any of Embodiments P-11 to P-16, wherein an intermediate prognostic score in the subject facilitates a determination by a medical practitioner of the need for one or more interventions selected further testing, proceeding with a cardiac catheterization or other dye-based procedure whereby dye usage is strictly limited, more frequent monitoring for risk factors, and lifestyle modifications.

Embodiment P-21. A panel for the prognosis of acute kidney injury comprising target-binding agents that bind at least two protein markers selected from those listed in Table 1, a synthetic standard, and optionally, at least one clinical variable selected from those listed in Table 2.

Embodiment P-22. A panel for the prognosis of acute kidney injury comprising target-binding agents for CD5 antigen-like, C-reactive protein, Factor VII, and osteopontin and the clinical variables of blood urea nitrogen:creatinine ratio and history of diabetes type 2.

Embodiment P-23. A panel for the prognosis of acute kidney injury risk comprising target-binding agents for CD5 antigen-like, C-reactive protein, Factor VII, kidney injury molecule 1, osteopontin and the clinical variables of blood urea nitrogen:creatinine ratio and history of diabetes type 2.

Embodiment P-24. A panel for the prognosis of acute kidney injury comprising target-binding agents for C-reactive protein, kidney injury molecule 1, and osteopontin and the clinical variables of blood urea nitrogen:creatinine ratio and history of diabetes type 2.

Embodiment P-25. A prognostic kit comprising a panel according to any one of Embodiments P-21 to P-24.

Embodiment P-26. Use of any of Embodiment P-21 to P-24 in the evaluation of a subject's positive, intermediate, or negative response to a therapeutic and/or intervention for acute kidney injury.

1. A method of determining risk of acute kidney injury in a subject, comprising:
(i) providing a biological sample from a subject suspected of having a risk of acute kidney injury,
(ii) applying the biological sample to an analytical device, to
(a) detect the concentration of at least two protein markers in the sample;
(b) normalize said concentration of protein markers against a synthetic quantification standard and
(c) transform the normalized protein marker concentrations;
wherein the at least two protein markers are selected from those set forth in Table 1;
(iii) optionally, determining the status of at least one clinical variable or measurement for the subject, wherein the clinical variable or measurement is selected from those set forth in Table 2;
(iv) calculating a score using an algorithm based on the normalized, transformed protein markers determined in step (ii) and, optionally, the status of the clinical variable or marker determined in step (iii);
(v) classifying the score as a positive, intermediate, or negative result; and
(vi) determining a prognosis of acute kidney injury risk in a subject as indicated by the score.

2. The method of claim 1, further comprising treating the subject based on the positive, intermediate, or negative score, wherein the treatment comprises a therapeutic intervention regimen.

3. The method of claim 1, wherein the sample comprises plasma.

4. The method of claim 1, wherein the at least two protein markers are selected from CD5 antigen like, C reactive protein, Factor VII, kidney injury molecule 1, N terminal prohormone of brain natriuretic peptide and osteopontin; and wherein the optional step (iii) comprises determining blood urea nitrogen:creatinine ratio and, optionally, the status of history of diabetes type 2.

5. The method of claim 1, wherein the at least two protein markers are C-reactive protein, CD5 antigen-like, Factor VII, and osteopontin, and wherein the optional step (iii) comprises determining blood urea nitrogen:creatinine ratio and the status of history of diabetes type 2.

6. The method of claim 1, wherein the at least two protein markers are C-reactive protein, CD5 antigen-like, kidney injury molecule 1, Factor VII, and osteopontin, and wherein the optional step (iii) comprises determining blood urea nitrogen:creatinine ratio and the status of history of diabetes type 2.

7. The method of claim 1, wherein the at least two protein markers are C-reactive protein, kidney injury molecule 1, and osteopontin, and wherein the optional step (iii) comprises determining blood urea nitrogen:creatinine ratio and the status of history of diabetes type 2.

8. The method of claim 1, wherein the at least two protein markers are C-reactive protein and N terminal prohormone of brain natriuretic peptide, and wherein the optional step (iii) comprises determining blood urea nitrogen:creatinine ratio.

9. The method of claim 1, wherein the prognosis of acute kidney injury risk in the subject comprises a prognosis of abrupt reduction in kidney function.

10. The method of claim 1, wherein a positive score in the subject facilitates a determination by a medical practitioner of the need for one or more interventions selected from ultrasound, administration of pharmacological agents, hydration, delaying a cardiac catheterization or other dye-based procedure and avoidance of any drug or procedure with a known kidney risk.

11. The method of claim 1, wherein a negative score in the subject facilitates a determination by a medical practitioner of the need for one or more interventions selected from ongoing monitoring and management of peripheral and coronary risk factors, and proceeding with a cardiac catheterization or other dye-based procedure.

12. The method of claim 1, wherein an intermediate score in the subject facilitates a determination by a medical practitioner of the need for one or more interventions selected from further testing, proceeding with a cardiac catheterization or other dye-based procedure whereby dye usage is strictly limited, more frequent monitoring for risk factors and lifestyle modifications.

13. A method of administering a therapeutic intervention to a subject having acute kidney injury risk comprising:
(i) determining the subject's protein marker profile for a panel of protein markers comprising at least two protein markers selected from those set forth in Table 1;
(ii) optionally, determining the status of at least one clinical variable or measurement for the subject, wherein the clinical variable or measurement is selected from those set forth in Table 2;
(iii) assigning a score to the subject based on the protein marker profile in (i) and optionally the clinical variable of measurement in (ii) wherein the score is classified as positive, intermediate, and negative, said score algorithmically-derived from the normalized and mathematically transformed concentrations of protein markers in the subject's sample and optionally, the status of at least one clinical variable or measurement; and
(iv) administering to the subject a therapeutic intervention based on the positive, intermediate or negative score.

14. A method of detecting two or more protein markers in a subject having diabetes type 2 and/or that is suspected of having acute kidney injury risk, the method comprising:
(i) selecting a subject that has diabetes type 2 and/or that is suspected of having acute kidney injury risk;
(ii) providing a biological sample from the subject;
(iii) applying the biological sample to an analytical device, and
(iv) detecting the concentration of at least two protein markers from Table 1.

15. The method of claim 14, further comprising:
(v) calculating a prognostic score based on the concentration of protein markers determined in step (iv);
(vi) classifying the prognostic score as a positive, intermediate, or negative result; and
(vii) determining acute kidney injury risk in a subject as indicated by the prognostic score.

16. The method of claim 13, wherein the at least two protein markers are selected from CD5 antigen like, C reactive protein, Factor VII, kidney injury molecule 1, N terminal prohormone of brain natriuretic peptide, and osteopontin and further wherein blood urea nitrogen:creatinine ratio is determined.

17. The method of claim 13, wherein the at least two protein markers are CD5 antigen-like, C-reactive protein, Factor VII, and osteopontin and further wherein blood urea nitrogen:creatinine ratio is determined.

18. The method of claim 13, wherein the at least two protein markers are CD5 antigen-like, C-reactive protein, Factor VII, kidney injury molecule 1, osteopontin and further wherein blood urea nitrogen:creatinine ratio is determined.

19. The method of claim 13, wherein the at least two protein markers are C-reactive protein, kidney injury molecule 1, and osteopontin and further wherein blood urea nitrogen:creatinine ratio is determined.

20. The method of claim 13, wherein the at least two protein markers are C-reactive protein and N terminal prohormone of brain natriuretic peptide and further wherein blood urea nitrogen:creatinine ratio is determined.

21. The method of claim 13, wherein the determination of acute kidney injury risk in the subject comprises a prognosis of abrupt reduction in kidney function.

22. The method of claim 13, wherein a positive prognostic score in the subject facilitates a determination by a medical practitioner of the need for one or more interventions selected from ultrasound, administration of pharmacological agents, hydration, delaying a cardiac catheterization or other dye-based procedure and avoidance of any drug or procedure with a known kidney risk.

23. The method of claim 13, wherein a negative prognostic score in the subject facilitates a determination by a medical practitioner of the need for one or more interventions selected from ongoing monitoring and management of peripheral and coronary risk factors, and proceeding with a cardiac catheterization or other dye-based procedure.

24. The method of claim 13, wherein an intermediate prognostic score in the subject facilitates a determination by a medical practitioner of the need for one or more interventions selected further testing, proceeding with a cardiac catheterization or other dye-based procedure whereby dye usage is strictly limited, more frequent monitoring for risk factors, and lifestyle modifications.

25. A panel for the prognosis of acute kidney injury comprising target-binding agents that bind at least two protein markers selected from those listed in Table 1, a synthetic standard, and optionally, at least one clinical variable selected from those listed in Table 2.

26-29. (canceled)

30. A prognostic kit comprising a panel according to claim 25.

31. (canceled)

32. (canceled)

33. A method of monitoring risk of acute kidney injury in a subject, comprising:
(i) providing a biological sample from a subject suspected of having a risk of acute kidney injury,
(ii) applying the biological sample to an analytical device, to
(a) detect the concentration of at least two protein markers in the sample;
(b) normalize said concentration of protein markers against a synthetic quantification standard and
(c) transform the normalized protein marker concentrations;
wherein the at least two protein markers are selected from those set forth in Table 1;
(iii) optionally, determining the status of at least one clinical variable or measurement for the subject, wherein the clinical variable or measurement is selected from those set forth in Table 2;
(iv) calculating a score using an algorithm based on the normalized, transformed protein markers determined in step (ii) and, optionally, the status of the clinical variable or marker determined in step (iii);
(v) classifying the score as a positive, intermediate, or negative result;
(vi) determining a prognosis of acute kidney injury risk in a subject as indicated by the score;
(vii) repeating steps (i) to (vi) from the same subject at a later timepoint; and
(viii) comparing the scores to determine if there is a change in acute kidney injury risk in the subject.
